(19) United States
(12) Patent Application Publication
(10) Pub. No.: US 2020/0157151 A1
(43) Pub. Date: May 21, 2020

(54) PEPTIDE COMPOUNDS, CONJUGATE COMPOUNDS AND USES THEREOF FOR TREATING INFLAMMATORY DISEASES

(71) Applicant: TRANSFERT PLUS, S.E.C., Montréal (CA)

(72) Inventors: Richard BÉLIVEAU, Montréal (CA); Borhane ANNABI, Brossard (CA); Michel DEMEULE, Beaconsfield (CA); Alain LAROCQUE, St-Laurent (CA); Jean-Christophe CURRIE, Repentigny (CA); Sylvie LAMY, Montréal (CA)

(73) Assignee: TRANSFERT PLUS, S.E.C., Montréal (CA)

(21) Appl. No.: 16/616,098

(22) PCT Filed: May 24, 2018
(86) PCT No.: PCT/CA2018/050606

§ 371 Date: Nov. 22, 2019

Related U.S. Application Data
(60) Provisional application No. 62/510,381, filed on May 24, 2017.

(51) Int. Cl. C07K 14/195; A61K 47/64; A61K 47/66; C07K 7/08; A61P 29/00; A61K 31/12

(52) U.S. Cl. CPC C07K 14/195; A61K 47/64; A61K 47/66; A61K 38/00; A61P 29/00; A61K 31/12; C07K 7/083

(57) ABSTRACT

The present disclosure relates to peptide compounds and conjugate compounds, processes, methods and uses thereof for treating inflammation. For example, the compounds can comprise compounds; IKLSGGVQAKAGVINMDKSESM, formula (V) as set forth in SEQ ID NO: 5, GVRAKAGVRN(Nle)FKSESY, formula (X) as set forth in SEQ ID NO: 10 and YKSLRRK.APRWDAPLRDPALRQLL, formula (XI) as set forth in SEQ ID NO: 11 wherein at least one protecting group and/or at least one labelling agent is connected to said peptide compound at an N- and/or C-terminal end, for use in inhibiting or decreasing TNF-alpha-induced COX-2 expression in cells expression sortilin.

### CROSS REFERENCE TO RELATED APPLICATIONS

The present application claims priority from U.S. provisional application No. 62/510,381 filed on May 24, 2017, which is hereby incorporated by reference in its entirety.

### FIELD OF THE DISCLOSURE

The present disclosure relates to peptide compounds and conjugate compounds and uses thereof for treating inflammation.

### BACKGROUND OF THE DISCLOSURE

Inflammation underlies a wide variety of physiological and pathological processes. Inflammation is the body's immediate response to damage to its tissues and cells by pathogens, noxious stimuli such as chemicals, or physical injury (Medzhitov 2008). Acute inflammation is a short-term response that usually results in healing: leukocytes infiltrate the damaged region, removing the stimulus and repairing the tissue. In contrast, chronic inflammation, is a prolonged, dysregulated and maladaptive response that involves active inflammation, tissue destruction and attempts at tissue repair. Such persistent inflammation is associated with many chronic human conditions and diseases, including allergy, atherosclerosis, cancer, obesity, arthritis and autoimmune diseases (Medzhitov 2008; Bradley 2007).

The global anti-inflammatory therapeutics market is expected to grow over the next few years, owing to the emergence of anti-inflammatory biologics that are more targeted, effective and with lesser side effects as compared to conventional drugs (from Global Anti-Inflammatory Therapeutics Market (2017-2020)). In addition, anti-inflammatory biologics are also difficult to reproduce due to their complex molecular structure and origin.

Traditionally, therapeutic approaches have sought to modulate the pro- or anti-inflammatory limbs of inflammation, with mixed success. For instance in oncology, insight into the pathways by which inflammation is resolved has highlighted novel opportunities to pharmacologically manipulate these processes that home in on specific molecular defects in cancer cells, promising more effective and less toxic therapies than imprecise therapeutic agents [Fisher et al., 2013].

Therefore, agents that are safe, cost effective and readily available are required.

### SUMMARY OF THE DISCLOSURE

Accordingly, a first aspect is a peptide compound having at least 80% sequence identity to a compound chosen from compounds of formula (I), formula (II), formula (III), formula (IV), formula (V), formula (VI), formula (VII), formula (VIII), formula (IX), formula (X), formula (XI) and formula (XII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid;
and wherein at least one protecting group and/or at least one labelling agent is optionally connected to said peptide at an N- and/or C-terminal end,
for use in treating inflammation.

In a further aspect disclosed herein is a conjugate compound having the formula of A-(B)ₙ,

wherein
n is 1, 2, 3 or 4;
A is a peptide compound as defined in the present disclosure, wherein said peptide is optionally protected by a protecting group; and
B is at least one therapeutic agent, wherein B is connected to A,
for use in treating treating inflammation.

In a further aspect disclosed herein is a conjugate compound having the formula of A-(B)ₙ,

wherein
n is 1, 2, 3 or 4;
A is a peptide compound as defined in the present disclosure, wherein said peptide is optionally protected by a protecting group; and
B is at least one therapeutic agent, wherein B is connected to A at a free amine of said peptide compound, at an N-terminal position of said peptide compound, at a free —SH of said peptide compound, or at a free carboxyl of said peptide compound,
for use in treating inflammation.

A further aspect disclosed herein is a conjugate compound having the formula of A-(B)ₙ,

wherein
n is 1, 2, 3 or 4;
A is a peptide compound as defined in the present disclosure, wherein said peptide is optionally protected by a protecting group; and
B is at least one therapeutic agent, wherein B is connected to A at a free amine of a lysine residue of said peptide compound, optionally via a linker, or at an N-terminal position of said peptide compound, optionally via a linker,
for use in treating inflammation.

Yet another aspect disclosed herein is sa conjugate compound having the formula (XV):

Acetyl-YK(curcumin)SLRRK(curcumin)APRWDAPLRDPALRQLL Formula (XV)
that comprises the peptide compound having SEQ ID NO: 16 wherein each lysine residue has a curcumin molecule connected thereto.

In an aspect, there is provided a process for preparing the conjugate compound disclosed in the present disclosure, the process comprising:
reacting a linker together with said at least one therapeutic agent so as to obtain an intermediate;
optionally purifying said intermediate;
reacting said intermediate together with said peptide compound so as to obtain said conjugate compound in which said at least one therapeutic agent is connected to said peptide compound via said linker; and
optionally purifying said conjugate compound;
wherein the at least one therapeutic agent is connected to the peptide compound at a free amine of a lysine residue or at an N-terminal; and wherein the peptide compound comprises 1, 2, 3 or 4 therapeutic agent molecules connected thereto.

In an aspect, there is provided a method of treating inflammation comprising administering to a subject in need thereof a therapeutically effective amount of at least one compound as defined herein.

In another aspect, there is provided a method of treating TNF-α-induced inflammation, comprising administering to a subject in need thereof a therapeutically effective amount of at least one compound as defined herein.

In another aspect, there is provided a method of treating inflammation in cells expressing Sortilin, comprising contacting said cells with at least one compound as defined herein.

In another aspect, there is provided a method of inhibiting TNF-α-induced COX-2 expression in cells expressing Sortilin, comprising contacting said cells with at least one compound as defined herein.

In another aspect, there is provided a method of decreasing TNF-α-induced COX-2 expression in cells expressing Sortilin, comprising contacting said cells with at least one compound as defined herein, wherein the TNF-α-induced COX-2 expression is decreased by at least about 20%, at least about 30%, at least about 40%, at least about 50%, at least about 60%, at least about 70%, at least about 80% or at least about 90%, about 5% to about 50%, about 10% to about 50%, about 15% to about 45%, about 20% to about 45% or about 30% to about 40%, greater than untreated cells expressing Sortilin.

In another aspect, there is provided a method of decreasing TNF-α-induced COX-2 expression in cells expressing Sortilin, comprising contacting said cells with at least one compound as defined herein, wherein the TNF-α-induced COX-2 expression is decreased by at least 1.2, at least 1.4, at least 1.6, at least 1.8, at least 2.0, at least 2.2, at least 2.4 fold, about 1.2 to about 2.4 fold or about 1.2 to about 2.0 fold greater than cells expressing Sortilin treated with the at least one therapeutic agent.

In another aspect, there is provided a method of inhibiting TNF-α-induced IκB phosphorylation in cells expressing Sortilin, comprising contacting said cells with at least one compound as defined herein.

In another aspect, there is provided a method of decreasing TNF-α-induced IκB phosphorylation in cells expressing Sortilin, comprising contacting said cells with at least one compound as defined herein, wherein the TNF-α-induced IκB phosphorylation is decreased by at least about 20%, at least about 30%, at least about 40%, at least about 50%, at least about 60%, at least about 70%, at least about 80% at least about 90%, about 5% to about 50%, about 10% to about 50%, about 15% to about 45%, about 20% to about 45% or about 30% to about 40%, greater than untreated cells expressing Sortilin.

In another aspect, there is provided a method of decreasing TNF-α-induced IκB phosphorylation in cells expressing Sortilin, comprising contacting said cells with at least one compound as defined herein, wherein the TNF-α-induced IκB phosphorylation is decreased by at least 1.2, at least 1.4, at least 1.6, at least 1.8, at least 2.0, at least 2.2, at least 2.4 fold, about 1.2 to about 2.4 fold or about 1.2 to about 2.0 fold greater than cells expressing Sortilin treated with the at least one therapeutic agent.

In another aspect, there is provided a method of increasing stability and/or bioavailability of a therapeutic agent, comprising:
obtaining the conjugate compound disclosed herein, wherein said conjugate compound comprises said therapeutic agent, and
administering a therapeutically effective amount of said conjugate compound to a subject in need thereof.

In another aspect, there is provided a method of increasing stability and/or bioavailability of a therapeutic agent, comprising:
conjugating said therapeutic agent with the peptide compound as defined herein to obtain a conjugate compound, and
administering a therapeutically effective amount of said conjugate compound to a subject in need thereof.

In another aspect, there is provided a use of at least one compound as defined herein for treating inflammation.

In another aspect, there is provided a use of at least one compound as defined herein for treating TNF-α-induced inflammation.

In another aspect, there is provided a use of at least one compound as defined herein for treating an inflammatory disease.

In another aspect, there is provided a use of at least one compound as defined herein for treating a TNF-α-induced inflammatory disease.

In another aspect, there is provided a use of at least one compound as defined herein for treating an inflammatory disease involving sortilin expression.

In another aspect, there is provided a use of at least one compound as defined herein for inhibiting TNF-α-induced COX-2 expression in cells expressing Sortilin.

In another aspect, there is provided a use of at least one compound as defined herein for decreasing TNF-α-induced COX-2 expression in cells expressing Sortilin by at least about 20%, at least about 30%, at least about 40%, at least about 50%, at least about 60%, at least about 70%, at least about 80% or at least about 90%, about 5% to about 50%, about 10% to about 50%, about 15% to about 45%, about 20% to about 45% or about 30% to about 40%, greater than untreated cells expressing Sortilin.

In another aspect, there is provided a use of at least one compound as defined herein for decreasing TNF-α-induced COX-2 expression in cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cells expressing Sortilin treated with the at least one therapeutic agent.

In another aspect, there is provided a use of at least one compound as defined herein for inhibiting TNF-α-induced IκB phosphorylation in cells expressing Sortilin.

In another aspect, there is provided a use of at least one compound as defined herein for decreasing TNF-α-induced IκB phosphorylation in cells expressing Sortilin by at least about 20%, at least about 30%, at least about 40%, at least about 50%, at least about 60%, at least about 70%, at least about 80%, at least about 90%, about 5% to about 50%, about 10% to about 50%, about 15% to about 45%, about 20% to about 45% or about 30% to about 40%, greater than untreated cells expressing Sortilin.

In another aspect, there is provided a use of at least one compound as defined herein for decreasing TNF-α-induced IκB phosphorylation in cells expressing Sortilin by at least 1.2, at least 1.4, at least 1.6, at least 1.8, at least 2.0, at least 2.2, at least 2.4 fold, about 1.2 to about 2.4 fold or about 1.2 to about 2.0 fold, greater than cells expressing Sortilin treated with the at least one therapeutic agent.

In another aspect, there is provided a use of a conjugate compound as defined herein for increasing stability and/or bioavailability of said at least one therapeutic agent.

In another aspect, there is provided a use of one compound as defined herein in the manufacture of a medicament for treating inflammation.

In another aspect, there is provided a use of one compound as defined herein in the manufacture of a medicament for treating TNF-α-induced inflammation.

In another aspect, there is provided a use of one compound as defined herein in the manufacture of a medicament for treating a TNF-α-induced inflammatory disease.

In another aspect, there is provided a use of one compound as defined herein in the manufacture of a medicament for treating an inflammatory disease involving sortilin expression.

In another aspect, there is provided a use of one compound as defined herein in the manufacture of a medicament for treating TNF-α-induced inflammation.

In another aspect, there is provided herein a method of increasing tolerability of a therapeutic agent, comprising:
conjugating the therapeutic agent with the peptide compound herein disclosed to obtain a conjugate compound, and
administering a therapeutically effective amount of the conjugate compound to a subject in need thereof.

In another aspect, there is provided herein a method of increasing tolerability of a therapeutic agent, comprising:
obtaining a conjugate compound herein disclosed, wherein the conjugate compound comprises the therapeutic agent, and
administering a therapeutically effective amount of the conjugate compound to a subject in need thereof.

For example, there is provided a use of a conjugate compound herein disclosed, for increasing tolerability of a therapeutic agent.

In a further aspect, there is provided a liposome, graphene, nanotube or nanoparticle comprising at least one compound as defined herein for use in treating inflammation.

In a yet another aspect, there is provided a liposome, graphene, nanotube or nanoparticle coated with at least one compound as defined herein for use in treating inflammation.

### BRIEF DESCRIPTION OF THE FIGURES

Further features and advantages of the disclosure will become more readily apparent from the following description of specific embodiments as illustrated by way of examples in the appended schemes and figures wherein:

FIG. 1 is a schematic of TNF-α-induced inflammatory cell signaling pathways.

FIG. 2 is a schematic of inflammatory targets modulated by Curcumin.

FIG. 3 is a series of Western blots showing Sortilin expression in cancer cell lines. The expression of Sortilin in various cancer cells was investigated by Western blotting. Immunoblots of 25 μg of protein per sample show that Sortilin is detected in most of the human cancer cell lines tested. In particular, high Sortilin levels were observed in many ovarian as well as in breast cancer cells, melanomas, colorectal, glioblastoma and hepatocellular adenocarcinoma.

FIG. 4 is a series of charts illustrating higher and sustained uptake of KBC-201 (FIG. 4A). At the same concentration (5 μM), KBC-201 generates about half the fluorescence compared to free curcumin (Ex.: 488 nm, Em.: 530 nm) (FIG. 4B). Time-course uptake of KBC-201 (full line) and free curcumin (dotted line) in human HT-29 colorectal cancer cells. HT-29 cells were incubated at 37° C. with 5 μM of KBC-201 or curcumin at various times, trypsinized, washed, and cell-associated fluorescence uptake was quantified using a BD Accuri™ C6 flow cytometer. KBC-201 shows a higher and sustained uptake over time compared to a transient uptake for free curcumin.

FIG. 5 is a series of charts showing Sortilin-mediated uptake of KBC-201 in human colorectal cancer cells, FIG. 5A) Uptake of 5 μM of KBC-201 or free curcumin were performed at 37° C. in control (siScrambled) or Sortilin-deficient (siSortilin) HT-29 colorectal cancer cells. After a 2 h incubation, cells were trypsinized to remove non-internalized products, washed 3-times with ice-cold PBS and cell-associated fluorescence was quantified using a BD Accuri™ C6 flow cytometer. The inhibition of Sortilin receptor (black bars) reduces KBC-201 uptake (left panel) but not that of free curcumin (right panel). FIG. 5B) The uptake of 5 μM of KBC-201 or free curcumin were also evaluated in HT-29 colorectal cancer cells in the absence (white bar) or presence (black bars) of excess unlabeled free-peptide (50 μM), neurotensin (10 μM) or progranulin (1 nM). Cell-associated fluorescence uptake was quantified using a BD Accuri™ C6 flow cytometer. Sortilin competitors inhibit the uptake of KBC-201 in HT-29 cells (left panel) but not that of free curcumin (right panel).

FIG. 6 shows the inhibition of TNF-α-induced COX-2 expression by Curcumin conjugate (KBC-201) in human HT-29 colon cancer cells. Cells were pre-treated for 2 h with indicated compounds in serum-free medium before the addition of 10 ng/mL TNF-α for 24 h. Cells were lysed and the levels protein expression of COX-2 were monitored by immunoblotting. FIG. 6A) Immunodetection of the induction of COX-2 protein expression by TNF-α is shown. FIG. 6B) The band intensities were analyzed by scanning densitometry using ImageJ software and the quantification is shown. For each sample, the COX-2 level was corrected for GAPDH (a loading control) and normalized to those seen in TNF-α control (value=100%).

FIG. 7 shows a comparison of Curcumin conjugates (KBC-106 and KBC-201) in inhibiting TNF-α-induced COX-2 expression in human HT-29 colon cancer cells. Cells were pre-treated for 2 h with indicated compounds in serum-free medium before the addition of 10 ng/mL TNF-α for 24 h. Cells were lysed and the protein expression levels of COX-2 were monitored by immunoblotting. FIG. 7A) Immunodetection of the induction of COX-2 protein expression by TNF-α is shown. FIG. 7B) The band intensities were analyzed by scanning densitometry using ImageJ software and the quantification is shown. For each sample, the COX-2 level was corrected for GAPDH (a loading control) and normalized to those seen in TNF-α control (value=100%).

FIG. 8 shows the inhibition of TNF-α-induced IκB phosphorylation by (KBC-201) in human HT-29 colon cancer cells. Cells were pre-treated for 24 h with indicated compounds in serum-free medium before the addition of 100 ng/mL TNF-α for 5 min. FIG. 8A) Immunodetection of IκB phosphorylation by TNF-α is shown. FIG. 8B) The band intensities were analyzed by scanning densitometry using ImageJ software and the quantification is shown. For each sample, IκB phosphorylation level was corrected for GAPDH (a loading control) and normalized to those seen in TNF-α control (value=100%).

FIG. 9 shows the inhibition of TNF-α-induced NFκB phosphorylation by Curcumin conjugate (KBC-201) in human MDA-MB231 breast cancer cells. Cells were pre-treated for 24 h with indicated compounds in serum-free medium before the addition of 100 ng/mL TNF-α for 5 min. FIG. 9A) Immunodetection of phosphorylated NFκB by TNF-α is shown. FIG. 9B) The band intensities were analyzed by scanning densitometry using ImageJ software and the quantification is shown. For each sample, the phosphorylated NFκB/non phosphorylated NFκB ratio was normalized to those seen in TNF-α control (value=100%).

FIGS. 10A and 10B show the inhibition of TNF-α-induced IκB phosphorylation by Curcumin conjugate (KBC-201) in human SKOV3 ovarian cancer cells. Cells were pre-treated for 24 h with indicated compounds in serum-free medium before the addition of 100 ng/mL TNF-α for 5 min. FIG. 10A) Immunodetection of IκB phosphorylation by TNF-α is shown. FIG. 10B) The band intensities were analyzed by scanning densitometry using ImageJ software and the quantification is shown. For each sample, IκB phosphorylation level was corrected for GAPDH (a loading control) and normalized to those seen in TNF-α control (value=100%).

FIG. 11 is a graph showing absorbance of Curcumin conjugates and free Curcumin. A better stability is shown for Curcumin conjugates than for free Curcumin. The absorbance of free Curcumin decreased more rapidly over time as compared to both Curcumin conjugates indicating that the conjugates are more stable. This suggests that the conjugation of Curcumin to Katana peptide(s) increases the stability of this phytochemical compound.

FIG. 12 is schematic representation of real time interaction analysis using Surface Plasmon Resonance (SPR) and a Biacore instrument.

FIGS. 13, 14, 15 and 16 show sensorgrams related to interactions of peptide compounds (KBP-106 in FIG. 13 and KBP-201 in FIG. 14) and Sortilin ligands (Receptor-Associated Protein (RAP) in FIG. 15 and Neurotensin in FIG. 16) with the Sortilin receptor using SPR.

FIGS. 17A and 17B show inhibition of TNF-α-induced IκB phosphorylation by Curcumin conjugate (KBC-201) in human HT-29 colon cancer cells.

FIGS. 18A and 18B show inhibition of TNF-α-induced NFκB phosphorylation by Curcumin conjugate (KBC-201) in human MDA-MB231 breast cancer cells.

### DETAILED DESCRIPTION OF THE DISCLOSURE

The term “peptide compounds” or “Katana peptides”, “Katana Biopharma Peptide” or “KBP” as used herein refers, for example, to peptides derived from bacterial proteins or from ligands of receptors that target receptors expressed on cancer cells including multidrug resistant cancer cells. For example, the peptide compounds can be derived from bacterial proteins involved in cell penetration or from sortilin ligands, for example progranulin and neurotensin. In certain embodiments, peptide compounds are connected (for example via a covalent bond, an atom or a linker) to at least one therapeutic agent (such as an anticancer agent or a phytochemical), thereby forming a conjugate compound that can be used, for example, for treating a cancer. In certain other embodiments, peptide compounds can be used at the surface of liposomes. For example, the peptide compounds can be used for coating liposomes, graphene, nanotubes or nanoparticles that can be loaded with at least one therapeutic agent (such as an anticancer agent or phytochemical, or genes or siRNA).

The term “Katana Biopharma Peptide Family 1 peptide compounds” or “KBP Family 1 peptide compounds” refers to peptide compounds derived from bacterial cell penetrant proteins. For example, KBP Family 1 peptide compounds can be derived from a protein having an amino acid sequence of IKLSGGVQAKAGVINMDKSESM (SEQ ID NO: 5). Non limiting examples of KBP Family 1 peptide compounds are shown below:

Amino acid sequences

| | |
|---|---|
| KBP-101 | IKLSGGVQAKAGVINMDKSESM-Formula (V) (represented by SEQ ID NO: 5) |
| KBP-102 | Succinyl-IKLSGGVQAKAGVINMFKSESY-Formula (XXXVI) (comprises SEQ ID NO: 6 |
| | wherein a succinyl group is attached at the N-terminal end) |
| KBP-103 | IKLSGGVQAKAGVINMFKSESYK(Biotin)-Formula (XXXVII) (comprises SEQ ID NO: 7 |
| | wherein a biotin molecule is connected thereto at the C-terminal end) |
| KBP-104 | GVQAKAGVINMFKSESY-Formula (VIII) (represented by SEQ ID NO: 8) |
| KBP-105 | Acetyl-GVRAKAGVRNMFKSESY-Formula (XXXVIII) (represented by SEQ ID NO: 14) |
| KBP-106 | Acetyl-GVRAKAGVRN(Nle)FKSESY-Formula (XXXIX) (represented by SEQ ID NO: 15) |

As used herein, the peptide compound KBP-101 is represented by the amino acid sequence of IKLSGGVQAKAGVINMDKSESM (SEQ ID NO: 5).

As used herein, the peptide compound KBP-102 is represented by the amino acid sequence of Succinyl-IKLSGGVQAKAGVINMFKSESY that comprises the peptide sequence of SEQ ID NO: 6 wherein a succinyl group is attached thereto at the N-terminal end.

As used herein, the peptide compound KBP-103 is represented by the amino acid sequence of IKLSGGVQAKAGVINMFKSESYK(Biotin) that comprises the peptide sequence of SEQ ID NO: 7 wherein a biotin molecule is connected thereto at the C-terminal end.

As used herein, the peptide compound KBP-104 is represented by the amino acid sequence of GVQAKAGVINMFKSESY (SEQ ID NO: 8).

As used herein, the peptide compound KBP-105 is represented by the amino acid sequence of Acetyl-GVRAKAGVRNMFKSESY (SEQ ID NO: 14).

As used herein, the peptide compound KBP-106 is represented by the amino acid sequence of Acetyl-GVRAKAGVRN(Nle)FKSESY (SEQ ID NO: 15).

The term “Katana Biopharma Peptide Family 2 peptide compounds” or “KBP Family 2 peptide compounds” refers to peptides derived from sortilin ligands, progranulin and neurotensin. For example, peptides can be derived from human, rat or mouse progranulin. For example, KBP Family 2 peptide compounds can be derived from human progranulin, for example having the amino acid sequence KCLRREAPRWDAPLRDPALRQLL (SEQ ID NO: 19), from rat progranulin, for example having the amino acid sequence KCLRKKTPRWDILLRDPAPRPLL (SEQ ID NO: 20), from mouse progranulin, for example having the amino acid sequence KCLRKKIPRWDMFLRDPVPRPLL (SEQ ID NO: 21), or from neurotensin, for example having an amino acid sequence XLYENKPRRPYIL (SEQ ID NO: 22). Non limiting examples of KBP Family 2 peptide compounds are shown below:

Amino acid sequences

| | |
|---|---|
| KBP-201 | Acetyl-YKSLRRKAPRWDAPLRDPALRQLL-Formula (XXXX) (represented by SEQ |
| | ID NO: 16) |
| KBP-202 | Acetyl-YKSLRRKAPRWDAYLRDPALRQLL-Formula (XXXXI) (represented by SEQ |
| | ID NO: 17) |
| KBP-203 | Acetyl-YKSLRRKAPRWDAYLRDPALRPLL-Formula (XXXXII) (represented by SEQ |
| | ID NO: 18) |

As used herein, the peptide compound KBP-201 is represented by the amino acid sequence of Acetyl-YKSLRRKAPRWDAPLRDPALRQLL (SEQ ID NO: 16).

As used herein, the peptide compound KBP-202 is represented by the amino acid sequence of Acetyl-YKSLRRKAPRWDAYLRDPALRQLL (SEQ ID NO: 17).

As used herein, the peptide compound KBP-203 is represented by the amino acid sequence of Acetyl-YKSLRRKAPRWDAYLRDPALRPLL (SEQ ID NO: 18).

The term “sortilin” as used herein refers to a neuronal type-1 membrane glycoprotein, encoded by the SORT1 gene, belonging to the Vacuolar Protein Sorting 10 protein (Vps10) family of receptors. Sortilin (also known as the neurotensin receptor 3) is expressed abundantly in the central and peripheral nervous systems and is also expressed in other types of tissues. For example, the expression of sortilin is upregulated in a number of cancers including for example ovarian, breast, colon and prostate cancer. Sortilin can exist in two forms, a full-length form (110 kDa) and a truncated form (95 kDa), corresponding to its large luminal domain (or ectodomain), which has been previously detected in the supernatant medium from sortilin-overexpressing cells (Navarro et al., 2002) The peptide compounds and conjugate compounds herein described can have a high binding affinity to sortilin and thus can specifically target cancer cells expressing or overexpressing sortilin.

The term “compound” as used in the present document refers to compounds of formulas (I), (II), (III), (IV), (V), (VI), (VII), (VIII), (IX), (X), (XI), (XII), (XIII), (XIV), (XV), or to pharmaceutically acceptable salts, solvates, hydrates and/or prodrugs of these compounds, isomers of these latter compounds, or racemic mixtures of these latter compounds, and/or to composition(s) made with such compound(s) as previously indicated in the present disclosure. The expression “compound” also refers to mixtures of the various compounds herein disclosed.

Compounds of the present disclosure include prodrugs. In general, such prodrugs will be functional derivatives of these compounds which are readily convertible in vivo into the compound from which it is notionally derived. Prodrugs of the compounds of the present disclosure may be conventional esters formed with available hydroxy, or amino group. For example, an available OH or nitrogen in a compound of the present disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the present disclosure are those in which one or more of the hydroxy groups in the compounds is masked as groups which can be converted to hydroxy groups in vivo. Conventional procedures for the selection and preparation of suitable prodrugs are described, for example, in “Design of Prodrugs” ed. H. Bundgaard, Elsevier, 1985.

Compounds of the present disclosure include radiolabeled forms, for example, compounds labeled by incorporation within the structure ²H, ³H, ¹⁴C, ¹⁵N, or a radioactive halogen such as ¹²⁵I. A radiolabeled compound of the compounds of the present disclosure may be prepared using standard methods known in the art.

The expression “derivative thereof” as used herein when referring to a compound means a derivative of the compound that has a similar reactivity and that could be used as an alternative to the compound in order to obtain the same desired result.

The term “inflammation” as used herein refers to an adverse immune response having a detrimental health effect in a subject. For example, it can refer to a reaction that occurs in affected cells and adjacent tissues in response to an injury, insult, abnormal stimulation caused by a physical, chemical, or biologic substance, or in response to ischemic conditions. For example, it can refer to a localized, protective response elicited by injury or destruction of tissues, which serves to destroy, dilute, or wall off (sequester) both the injurious agent and the injured tissue. Inflammation can be associated with influx of leukocytes and/or neutrophil chemotaxis. For example, it can refer to the definition of “inflammation” as provided in http://medical-dictionary.thefreedictionary.com/Inflammation, which is hereby incorporated by reference.

The expression “inflammatory disease” as used herein refers to any disease, disorder, or syndrome in which an excessive or unregulated inflammatory response leads to excessive inflammatory symptoms, host tissue damage, or loss of tissue function. This expression can also refer to a pathological state mediated by influx of leukocytes and/or neutrophil chemotaxis.

The expression “therapeutic agent” as used herein means and agent capable of producing a therapeutic effect by inhibiting or decreasing inflammation in a subject or in cells, compared to a control. For example, the therapeutic agent is an anti-inflammatory agent such as a phytochemical, a non-steroidal anti-inflammatory drug, a steroidal anti-inflammatory drug, an antileukotrine agent, a biologic agent or an immune-selective anti-inflammatory derivative (ImSAID).

The term “phytochemical” as used herein means chemical compounds that occur naturally in plants and that can be used for treating inflammation. Examples of phytochemicals include for example Curcumin. Curcumin (diferuloylmethane) is a yellow pigment present in the spice turmeric (Curcuma longa) that has been associated with anti-inflammatory. Other phytochemicals with anti-inflammatory properties include for example omega-3, white willow bark, green tea, catechins, pycnogenol, Boswellia serrata resin, resveratrol, Uncaria tomentosa, capsaicin, anthocyanins/anthocyanidins, flavanoids, olive oil compounds, chlorogenic acid and sulfopharaphane.

The term “curcumin” or “cur” as used herein means a phytochemical having the structure:

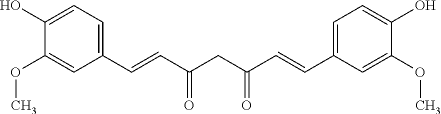 [SMILES: COc1cc(/C=C/C(=O)CC(=O)/C=C/c2ccc(O)c(OC)c2)ccc1O]

or pharmaceutically acceptable salts, solvates or prodrugs thereof as well as mixtures thereof. For example, curcumin can be conjugated to a peptide compound of the present disclosure via an oxygen atom of its phenol groups. Curcumin can be connected to the peptide compound directly or via a linker.

The expression “conjugate compounds” or “peptide-drug conjugates” as used herein refers to compounds comprising a peptide compound herein disclosed connected to at least one therapeutic agent, optionally via a linker. Conjugate compounds can comprise, for example, 1, 2, 3 or 4 molecules of a therapeutic agent connected thereto. These 1-4 molecules of therapeutic agent can be the same or different i.e. up to four different therapeutic agents could be connected to the peptides. The therapeutic agent(s) are connected to the peptide via at least one covalent bond, at least one atom or at least one linker. Conjugate compounds can be used in the treatment of inflammation. Examples of conjugate compounds include, without limitation, the conjugate compounds shown below:

| | |
|---|---|
| KBC-106 (2:1) | Acetyl-GVRAK(curcumin)AGVRN(Nle)FK(curcumin)SESY-Formula (XIV) |
| | that comprises the peptide compound having SEQ ID NO: 15 wherein each lysine |
| | residue has a curcumin molecule connected thereto |
| KBC-201 (2:1) | Acetyl-YK(curcumin)SLRRK(curcumin)APRWDAPLRDPALRQL-Formula (XV) |
| | that comprises the peptide compound having SEQ ID NO: 16 wherein each lysine |
| | residue has a curcumin molecule connected thereto |

The term “conjugating” au used herein, refers, for example, to the preparation of a conjugate as defined above. Such an action comprises connecting a peptide compound together with at least one therapeutic agent, optionally via a linker.

For example, the following are general chemical formulas of some conjugate compounds herein disclosed.

Curcumin-Katana peptide conjugate:

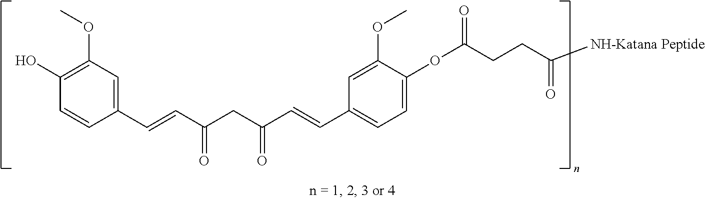

For example, the following are the chemical structures of some conjugate compounds herein disclosed.

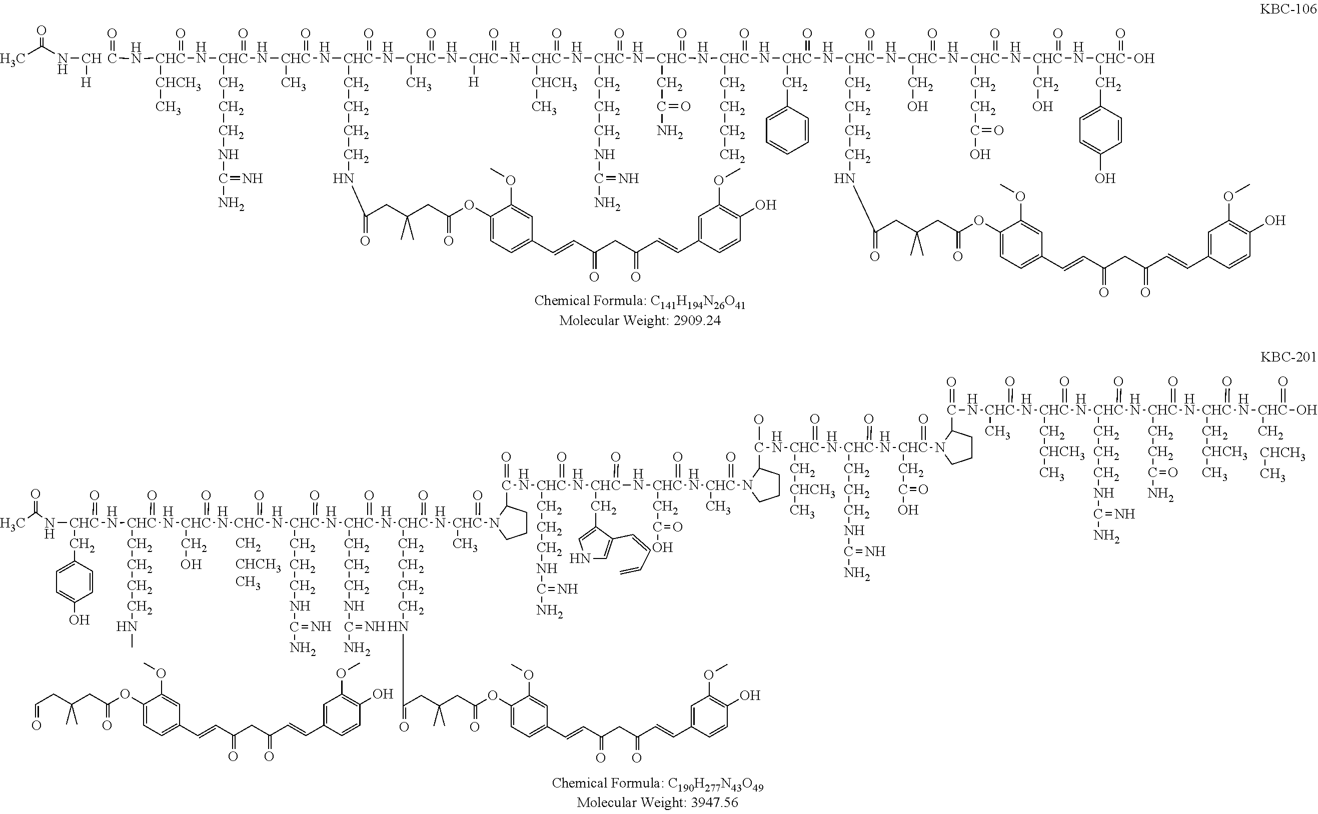

The term “linker” as used herein means a chemical structure connecting a peptide compound herein disclosed to at least one therapeutic agent. The linker can be primary amines (amines (—NH2): this group exists at the N-terminus of each polypeptide epsilon-amine). For example, the linker can be connected to the peptide compound at the carboxyls (—COOH): this group exists at the C-terminus of each polypeptide chain and in peptide compound herein disclosed to at least one therapeutic agent. The linker can be connected to the peptide compound at different functional groups on the peptide compounds. For example, the linker can be connected to the peptide compound at the primary amines (amines (—NH2): this group exists at the N-terminus of each polypeptide chain (called the alpha-amine) and in the side chain of lysine (Lys, K) residues (called the epsilon-amine). For example, the linker can be connected to the peptide compound at the carboxyls (—COOH): this group exists at the C-terminus of each polypeptide chain and in the side chains of aspartic acid (Asp, D) and glutamic acid (Glu, E). For example, the linker can be connected to the peptide compound at the Sulfhydryls (—SH): This group exists in the side chain of cysteine (Cys, C). Often, as part of a protein's secondary or tertiary structure, cysteines are joined together between their side chains via disulfide bonds (—S—S—). These must be reduced to sulfhydryls to make them available for crosslinking by most types of reactive groups. For example, the linker can be connected to the peptide compound at the Carbonyls (—CHO): Ketone or aldehyde groups can be created in glycoproteins by oxidizing the polysaccharide post-translational modifications (glycosylation) with sodium meta-periodate. For example, the linker can be a cleavable linker. For example, the linker can be a non-cleavable linker.

The following table summarizes the reactivity class and the chemical group of some of the principals linkers for standard chemical conjugation:

| Reactivity class | Chemical group |
|---|---|
| Carboxyl-to-amine reactive groups | Carbodiimide (e.g., EDC) |
| Amine-reactive groups | NHS ester |
| | Imidoester |
| | Pentafluorophenyl ester |
| | Hydroxymethyl phosphine |
| Sulfhydryl-reactive groups | Maleimide |
| | Haloacetyl (Bromo- or Iodo-) |
| | Pyridyldisulfide |
| | Thiosulfonate |
| | Vinylsulfone |
| Aldehyde-reactive groups | Hydrazide |
| i.e., oxidized sugars (carbonyls) | Alkoxyamine |
| Photoreactive groups | Diazirine |
| | Aryl Azide |

For example, homobifunctional and heterobifunctional crosslinkers can be used. For example, Disuccinimidyl suberate (DSS) is a homobifunctional crosslinker that has identical amine-reactive NHS-ester groups at either end of a short spacer arm. For example, Sulfosuccinimidyl 4-(N-maleimidomethyl)cyclohexane-1-carboxylate (Sulfo-SMCC) is a heterobifunctional crosslinker that has an amine-reactive sulfo-NHS-ester group at one end and a sulfhydryl reactive maleimide group at the opposite end of a cyclohexane spacer arm. This allows for sequential, two-step conjugation procedures. Among the commercially available homobifunctional cross-linkers are: BSOCOES (Bis(2-[Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); and BASED (Bis(β-[4-azidosalicylamido]-ethyl)disulfide iodinatable).

The polypeptides may be conjugated through a variety of linkers, e.g., sulfhydryl groups, amino groups (amines), or any appropriate reactive group. The linker can be a covalent bond. The linker group may comprise a flexible arm, e.g., 2, 3, 4, 5, 6, 7, 8, 9, 10, 11, 12, 13, 14, or 15 carbon atoms.

Exemplary linkers include, without limitation, pyridinedisulfide, thiosulfonate, vinylsulfonate, isocyanate, imidoester, diazine, hydrazine, thiol, carboxylic acid, multi-peptide linkers, and acetylene. Alternatively other linkers that can be used include BS³ [Bis(sulfosuccinimidyl)suberate] (which is a homobifunctional N-hydroxysuccinimide ester that targets accessible primary amines), NHS/EDC (N-hydroxysuccinimide and 1-ethyl-3-(3-dimethylaminopropyl)carbodiimide (NHS/EDC allows for the conjugation of primary amine groups with carboxyl groups), sulfo-EMCS ([N-∈-maleimidocaproic acid]hydrazide (sulfo-EMCS are heterobifunctional reactive groups that are reactive toward sulfhydryl and amino groups), hydrazide (most proteins contain exposed carbohydrates and hydrazide is a useful reagent for linking carboxyl groups to primary amines).

To form covalent bonds, one can use as a chemically reactive group a wide variety of active carboxyl groups (e.g., esters) where the hydroxyl moiety is physiologically acceptable at the levels required to modify the peptide. Particular agents include for example N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA), maleimido hexanoic acid (MHA), and maleimido undecanoic acid (MUA).

Primary amines are the principal targets for NHS esters; NHS esters react with primary amines to form covalent amide bonds. Accessible α-amine groups present on the N-termini of proteins and the ε-amine of lysine react with NHS esters. Thus, conjugated compounds herein disclosed can include a linker having a NHS ester conjugated to an N-terminal amino of a peptide or to an ε-amine of lysine. An amide bond is formed when the NHS ester reacts with primary amines releasing N-hydroxysuccinimide. Succinimide containing reactive groups may be referred to more simply as succinimidyl groups. In some embodiments, the functional group on the protein will be a thiol group and the chemically reactive group will be a maleimido-containing group such as gamma-maleimide-butylamide (GMBA or MPA). Such maleimide-containing groups may be referred to herein as maleido groups.

Amine-to-amine linkers include NHS esters, imidoesters, and others, examples of which are listed below.

### Exemplary NHS Esters:

DSG (disuccinimidyl glutarate)
DSS (disuccinimidyl suberate)
BS³ (bis[sulfosuccinimidyl] suberate)
TSAT (tris-succinimidyl aminotriacetate)
Variants of bis-succinimide ester-activated compounds including a polyethylene glycol spacer such as BS(PEG)ₙ where n is 1-20 (e.g., BS(PEG)₅ and BS(PEG)₉)
DSP (Dithiobis[succinimidyl propionate])
DTSSP (3,3′-dithiobis[sulfosuccinimidylpropionate])
DST (disuccinimidyl tartarate)
BSOCOES (bis[2-(succinimidooxycarbonyloxy)ethyl]sulfone)
EGS (ethylene glycol bis[succinimidylsuccinate])
sulfo-EGS (ethylene glycol bis[sulfosuccinimidylsuccinate])

### Exemplary Imidoesters:

DMA (dimethyl adipimidate.2HCl)
DMP (dimethyl pimelimidate.2HCl)
DMS (dimethyl suberimidate.2HCl)
DTBP (dimethyl 3,3′-dithiobispropionimidate.2HCl)

### Other Exemplary Amine-to-Amine Linkers:

DFDNB (1,5-difluoro-2,4-dinitrobenzene)
THPP (β-[tris(hydroxymethyl) phosphino] propionic acid (betaine))

The linker may also be a sulfhydryl-to-sulfhydryl linker, such as the maleimides and pyridyldithiols listed below.

Exemplary maleimides:
BMOE (bis-maleimidoethane)
BMB (1,4-bismaleimidobutane)
BMH (bismaleimidohexane)
TMEA (tris[2-maleimidoethyl]amine)
BM(PEG)2 1,8-bis-maleimidodiethyleneglycol)
BM(PEG)ₙ, where n is 1 to 20 (e.g., 2 or 3)
BMDB (1,4 bismaleimidyl-2,3-dihydroxybutane)
DTME (dithio-bismaleimidoethane)
Exemplary pyridyldithiol:
DPDPB (1,4-di-[3′-(2′-pyridyldithio)-propionamido]butane)
Another sulfhydryl linker:
HBVS (1,6-hexane-bis-vinylsulfone)

The linker may be an amine-to-sulfhydryl linker, which includes NHS ester/maliemide compounds. Examples of these compounds are provided below.

### Amine-to-Sulfhydryl Linkers:

AMAS (N-(α-maleimidoacetoxy)succinimide ester)
BMPS (N-[β-maleimidopropyloxy]succinimide ester)
GMBS (N-[γ-maleimidobutyryloxy]succinimide ester)
sulfo-GMBS (N-[γ-maleimidobutyryloxy]sulfosuccinimide ester)
MBS (m-maleimidobenzoyl-N-hydroxysuccinimide ester)
sulfo-MBS (m-maleimidobenzoyl-N-hydroxysulfosuccinimide ester)
SMCC (succinimidyl 4-[N-maleimidomethyl]cyclohexane-1-carboxylate)
sulfo-SMCC (Sulfosuccinimidyl 4-[N-maleimidomethyl]cyclohexane-1-carboxylate)
EMCS ([N-ε-maleimidocaproyloxy]succinimide ester)
Sulfo-EMCS ([N-ε-maleimidocaproyloxy]sulfosuccinimide ester)
SMPB (succinimidyl 4-[p-maleimidophenyl]butyrate)
sulfo-SMPB (sulfosuccinimidyl 4-[p-maleimidophenyl]butyrate)
SMPH (succinimidyl-6-[β-maleimidopropionamido]hexanoate)
LC-SMCC (succinimidyl-4-[N-maleimidomethyl]cyclohexane-1-carboxy-[6-amidocaproate])
sulfo-KMUS (N-[κ-maleimidoundecanoyloxy]sulfosuccinimide ester)
SM(PEG)ₙ (succinimidyl-([N-maleimidopropionamido-polyethyleneglycol) ester), where n is 1 to 30 (e.g., 2, 4, 6, 8, 12, or 24)
SPDP (N-succinimidyl 3-(2-pyridyldithio)-propionate)
LC-SPDP (succinimidyl 6-(3-[2-pyridyldithio]-propionamido)hexanoate)
sulfo-LC-SPDP (sulfosuccinimidyl 6-(3′-[2-pyridyldithio]-propionamido)hexanoate)
SMPT (4-succinimidyloxycarbonyl-α-methyl-α-[2-pyridyldithio]toluene)
Sulfo-LC-SMPT (4-sulfosuccinimidyl-6-[α-methyl-α-(2-pyridyldithio)toluamido]hexanoate)
SIA (N-succinimidyl iodoacetate)
SBAP (succinimidyl 3-[bromoacetamido]propionate)
SIAB (N-succinimidyl[4-iodoacetyl]aminobenzoate)
sulfo-SIAB (N-sulfosuccinimidyl[4-iodoacetyl]aminobenzoate)

The linker can react with an amino group and a non-selective entity. Such linkers include NHS ester/aryl azide and NHS ester/diazirine linkers, examples of which are listed below.

### NHS Ester/Aryl Azide Linkers:

NHS-ASA (N-hydroxysuccinimidyl-4-azidosalicylic acid)
ANB-NOS (N-5-azido-2-nitrobenzoyloxysuccinimide)
sulfo-HSAB (N-hydroxysulfosuccinimidyl-4-azidobenzoate)
sulfo-NHS-LC-ASA (sulfosuccinimidyl[4-azidosalicylamido]hexanoate)
SANPAH (N-succinimidyl-6-(4′-azido-2′-nitrophenylamino)hexanoate)
sulfo-SANPAH (N-sulfosuccinimidyl-6-(4′-azido-2′-nitrophenylamino)hexanoate)
sulfo-SFAD (sulfosuccinimidyl-(perfluoroazidobenzamido)-ethyl-1,3′-dithioproprionate)
sulfo-SAND (sulfosuccinimidyl-2-(m-azido-o-nitrobenzamido)-ethyl-1,3′-proprionate)
sulfo-SAED (sulfosuccinimidyl 2-[7-amino-4-methylcoumarin-3-acetamido]ethyl-1,3′dithiopropionate)

### NHS Ester/Diazirine Linkers:

SDA (succinimidyl 4,4′-azipentanoate)
LC-SDA (succinimidyl 6-(4,4′-azipentanamido)hexanoate)
SDAD (succinimidyl 2-([4,4′-azipentanamido]ethyl)-1,3′-dithioproprionate)
sulfo-SDA (sulfosuccinimidyl 4,4′-azipentanoate)
sulfo-LC-SDA (sulfosuccinimidyl 6-(4,4′-azipentanamido)hexanoate)
sulfo-SDAD (sulfosuccinimidyl 2-([4,4′-azipentanamido]ethyl)-1,3′-dithioproprionate)

Exemplary amine-to-carboxyl linkers include carbodiimide compounds (e.g., DCC (N,N-dicyclohexylcarbodimide) and EDC (1-ethyl-3-[3-dimethylaminopropyl]carbodiimide)). Exemplary sulfhydryl-to-nonselective linkers include pyridyldithiol/aryl azide compounds (e.g., APDP ((N-[4-(p-azidosalicylamido)butyl]-3′-(2′-pyridyldithio)propionamide)). Exemplary sulfhydryl-to-carbohydrate linkers include maleimide/hydrazide compounds (e.g., BMPH (N-[(3-maleimidopropionic acid]hydrazide), EMCH ([N-ε-maleimidocaproic acid]hydrazide), MPBH 4-(4-N-maleimidophenyl)butyric acid hydrazide), and KMUH (N-[κ-maleimidoundecanoic acid]hydrazide)) and pyridyldithiol/hydrazide compounds (e.g., PDPH (3-(2-pyridyldithio)propionyl hydrazide)). Exemplary carbohydrate-to-nonselective linkers include hydrazide/aryl azide compounds (e.g., ABH (p-azidobenzoyl hydrazide)). Exemplary hydroxyl-to-sulfhydryl linkers include isocyanate/maleimide compounds (e.g., (N-[p-maleimidophenyl]isocyanate)). Exemplary amine-to-DNA linkers include NHS ester/psoralen compounds (e.g., SPB (succinimidyl-[4-(psoralen-8-yloxy)]-butyrate)).

To generate a branch point of varying complexity in a conjugate peptide, the linker can be capable of linking 3-7 entities.

Exemplary tri-functional linkers:
TMEA; Tris-(2-
maleimidoethyl)amine)

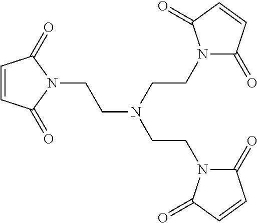 [SMILES: O=C1C=CC(=O)N1CCN(CCN1C(=O)C=CC1=O)CCN1C(=O)C=CC1=O]

THPP

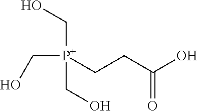 [SMILES: O=C(O)CC[P+](CO)(CO)CO]

LC-TSAT (tris-succinimidyl (6-
aminocaproyl)aminotriacetate), tris-
succinimidyl-1,3,5-benzenetricarboxylate
MDSI (maleimido-3,5-disuccinimidyl
isophthalate)
TSAT; Tris-succinimidyl aminotriacetate

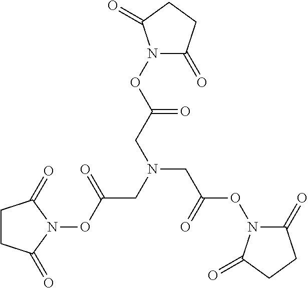 [SMILES: O=C(CN(CC(=O)ON1C(=O)CCC1=O)CC(=O)ON1C(=O)CCC1=O)ON1C(=O)CCC1=O]

SDMB (succinimidyl-3,5-
dimaleimidophenyl benzoate
Mal-4 (tetrakis-(3-maleimidopropyl)
pentaerythritol, NHS-4 (tetrakis-(N-
succinimidylcarboxypropyl)pentaerythritol))

TMEA and TSAT reach through their maleimide groups with sulfhydryl groups. The hydroxyl groups and carboxy group of THPP can react with primary or secondary amines. Other useful linkers conform to the formula Y═C═N-Q-A-C(O)—Z, where Q is a homoaromatic or heteroaromatic ring system; A is a single bond or an unsubstituted or substituted divalent C₁₋₃₀ bridging group, Y is O or S; and Z is Cl, Br, I, N₃, N-succinimidyloxy, imidazolyl, 1-benzotriazolyloxy, OAr where Ar is an electron-deficient activating aryl group, or OC(O)R where R is -A-Q-N═C═Y or C₄-20 tertiary-alkyl (see U.S. Pat. No. 4,680,338).

Other useful linkers have the formula

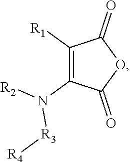

where R₁ is H, C₁₋₆ alkyl, C₂₋₆ alkenyl, C₆₋₁₂ aryl or aralkyl or these coupled with a divalent organic —O—, —S—, or

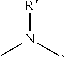

where R′ is C₁₋₆ alkyl, linking moiety; R₂ is H, C₁₋₁₂ alkyl, C₆₋₁₂ aryl, or C₆₋₁₂ aralkyl, R₃ is

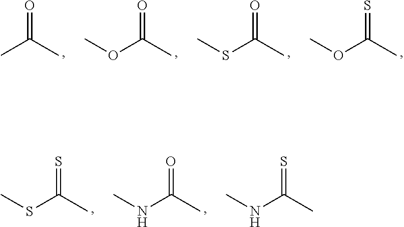

or another chemical structure that is able to delocalize the lone pair electrons of the adjacent nitrogen and R₄ is a pendant reactive group capable of linking R₃ to a peptide vector or to an agent (see for example U.S. Pat. No. 5,306,809).

The linker may include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly or Cys). Where the linker is a short peptide, it can be a glycine-rich peptide (which tend to be flexible) such as a peptide having the sequence [Gly-Gly-Gly-Gly-Ser]ₙ where n is an integer from 1 to 6, inclusive (see U.S. Pat. No. 7,271,149) or a serine-rich peptide linker (see U.S. Pat. No. 5,525,491). Serine rich peptide linkers include those of the formula [X-X-X-X-Gly]_{y} where up to two of the X are Thr, the remaining X are Ser, and y is an integer from 1 to 5, inclusive (e.g., Ser-Ser-Ser-Ser-Gly, where y is greater than 1). Other linkers include rigid linkers (e.g., PAPAP and (PT)ₙP, where n is 2, 3, 4, 5, 6, or 7) and α-helical linkers (e.g., A(EAAAK)ₙA, where n is 1, 2, 3, 4, or 5).

The linker can be an aliphatic linker (e.g., with an amide bond to the polypeptide and an ester bond to the therapeutic agent). Where an aliphatic linker is used, it may vary with regard to length (e.g. C₁-C₂₀) and the chemical moieties it includes (e.g., an amino group or carbamate).

Examples of suitable amino acid linkers are succinic acid, Lys, Glu, and Asp, or a dipeptide such as Gly-Lys. When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

The linker can also be a branched polypeptide. Exemplary branched peptide linkers are described in U.S. Pat. No. 6,759,509.

The linker can provide a cleavable linkage (e.g., a thioester linkage) or a non-cleavable linkage (e.g., a maleimide linkage). For example, a cytotoxic protein can be bound to a linker that reacts with modified free amines, which are present at lysine residues within the polypeptide and at the amino-terminus of the polypeptide. Thus, linkers useful in the present conjugate compounds can comprise a group that is reactive with a primary amine on the polypeptide or modified polypeptide to which the therapeutic agent moiety is conjugated. More specifically, the linker can be selected from the group consisting of monofluoro cyclooctyne (MFCO), bicyclo[6.1.0]nonyne (BCN), N-succinimidyl-S-acetylthioacetate (SATA), N-succinimidyl-S-acetylthiopropionate (SATP), maleimido and dibenzocyclooctyne ester (a DBCO ester). Useful cyclooctynes, within a given linker, include OCT, ALO, MOFO, DIFO, DIBO, BARAC, DIBAC, and DIMAC.

The linker may comprise a flexible arm, such as for example, a short arm (<2 carbon chain), a medium-size arm (from 2-5 carbon chain), or a long arm (3-6 carbon chain).

Click chemistry can also be used for conjugation on a peptide (DBCO, TCO, tetrazine, azide and alkyne linkers). These families of linkers can be reactive toward amine, carboxyl and sulfhydryl groups. In addition, these linkers can also be biotinylated, pegylated, modified with a fluorescent imaging dye, or phosphoramidited for incorporation onto an oligonucleotide sequence.

The term “intermediate” as used herein refers to a therapeutic agent that has been reacted with a linker thereby forming an intermediate or an activated form of the therapeutic agent. The intermediate can be reacted with a peptide compound herein disclosed thereby forming a conjugate compound herein disclosed that can be used for treating a cancer.

The expression “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “non-standard,” “unnatural” or “unusual” refers to the wide selection of non-natural, rare or synthetic amino acids such as those described by Hunt, S. in Chemistry and Biochemistry of the Amino Acids, Barrett, G. C., ed., Chapman and Hall: New York, 1985. Some examples of non-standard amino acids include non-alpha amino acids, D-amino acids.

Abbreviations used for amino acids and designation of peptides follow the rules of the IUPAC-IUB Commission of Biochemical Nomenclature in J. Biol. Chem. 1972, 247, 977-983. This document has been updated: Biochem. J., 1984, 219, 345-373; Eur. J. Biochem., 1984, 138, 9-37; 1985, 152, 1; Int. J. Pept. Prot. Res., 1984, 24, following p 84; J. Biol. Chem., 1985, 260, 14-42; Pure Appl. Chem. 1984, 56, 595-624; Amino Acids and Peptides, 1985, 16, 387-410; and in Biochemical Nomenclature and Related Documents, 2^{nd} edition, Portland Press, 1992, pp 39-67. Extensions to the rules were published in the JCBN/NC-IUB Newsletter 1985, 1986, 1989; see Biochemical Nomenclature and Related Documents, 2^{nd} edition, Portland Press, 1992, pp 68-69.

The term “antagonist” refers to a compound that reduces at least some of the effect of the endogenous ligand of a protein, receptor, enzyme, interaction, or the like.

The term “inhibitor” refers to a compound that reduces the normal activity of a protein, receptor, enzyme, interaction, or the like.

The expression “inverse agonist” refers to a compound that reduces the activity of a constitutively-active receptor below its basal level.

The term “library” refers to a collection of compounds that can be used for example for drug discovery purposes. For example, the library compounds can be peptide compounds and/or conjugate compounds herein disclosed.

The term “mixture” as used herein, means a composition comprising two or more compounds. In an embodiment a mixture is a mixture of two or more distinct compounds. In a further embodiment, when a compound is referred to as a “mixture”, this means that it can comprise two or more “forms” of the compounds, such as, salts, solvates, prodrugs or, where applicable, stereoisomers of the compound in any ratio. A person of skill in the art would understand that a compound in a mixture can also exist as a mixture of forms. For example, a compound may exist as a hydrate of a salt or as a hydrate of a salt of a prodrug of the compound. All forms of the compounds disclosed herein are within the scope of the present application.

The term “modulator” refers to a compound that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist.” Exemplary biological processes or mechanisms affected by a modulator include, but are not limited to, enzyme binding, receptor binding and hormone release or secretion. Exemplary chemical processes or mechanisms affected by a modulator include, but are not limited to, catalysis and hydrolysis.

The term “peptide” refers to a chemical compound comprising at least two amino acids covalently bonded together using amide bonds.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary and not exhaustive, and one skilled in the art could prepare other known varieties of prodrugs. Prodrugs may be, for example, formed with available hydroxy, thiol, amino or carboxyl groups. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The expression “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in Protective Groups in Organic Synthesis, T. W. Greene and P. G. Wuts, eds., John Wiley & Sons, New York, 4^{th} edition, 2006, 1082 pp, ISBN 9780471697541. Examples of amino protecting groups include, but are not limited to, phthalimido, trichloroacetyl, benzyloxycarbonyl, tert butoxycarbonyl, and adamantyl-oxycarbonyl. In some embodiments, amino protecting groups are carbamate amino protecting groups, which are defined as an amino protecting group that when bound to an amino group forms a carbamate. In other embodiments, amino carbamate protecting groups are allyloxycarbonyl (Alloc), benzyloxycarbonyl (Cbz), 9 fluorenylmethoxycarbonyl (Fmoc), tert-butoxycarbonyl (Boc) and α,α dimethyl-3,5 dimethoxybenzyloxycarbonyl (Ddz). For a recent discussion of newer nitrogen protecting groups see: Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to, methyl ester, tert-butyl ester, benzyl ester, trimethylsilylethyl ester, and 2,2,2-trichloroethyl ester.

The expression “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The expression “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The expression “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry (Solid-Phase Synthesis: A Practical Guide, F. Albericio, ed., CRC Press, 2000, 848 pp, ISBN: 978-0824703592; Organic Synthesis on Solid Phase, 2^{nd} edition, Florencio Zaragoza Dorwald, Wiley-VCH, 2002, 530 pp, ISBN: 3-527-30603-X; Solid-Phase Organic Synthesis: Concepts, Strategies, and Applications, P. H. Toy, Y. Lam, eds., Wiley, 2012, 568 pp, ISBN: 978-0470599143).

The term “solid support,” “solid phase” or “resin” refers to a mechanically and chemically stable polymeric matrix utilized to conduct solid phase chemistry. This is denoted by “Resin,” “P-” or the following symbol:

[symbol]

Examples of appropriate polymer materials include, but are not limited to, polystyrene, polyethylene, polyethylene glycol (PEG, including, but not limited to, ChemMatrix® (Matrix Innovation, Quebec, Quebec, Canada; J. Comb. Chem. 2006, 8, 213-220)), polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., ed.; SPCC Ltd.: Birmingham, UK; p 205), polyacrylate (CLEAR™), polyacrylamide, polyurethane, PEGA [polyethyleneglycol poly(N,N dimethyl-acrylamide) co-polymer, Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, preferably 0.5-2%). This solid support can include as non-limiting examples aminomethyl polystyrene, hydroxymethyl polystyrene, benzhydrylamine polystyrene (BHA), methylbenzhydrylamine (MBHA) polystyrene, and other polymeric backbones containing free chemical functional groups, most typically, NH₂ or —OH, for further derivatization or reaction. The term is also meant to include “Ultraresins” with a high proportion (“loading”) of these functional groups such as those prepared from polyethyleneimines and cross-linking molecules (J. Comb. Chem. 2004, 6, 340-349). At the conclusion of the synthesis, resins are typically discarded, although they have been shown to be able to be recycled (Tetrahedron Lett. 1975, 16, 3055).

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The expression “pharmaceutically acceptable” means compatible with the treatment of subjects such as animals or humans.

The expression “pharmaceutically acceptable salt” means an acid addition salt or basic addition salt which is suitable for or compatible with the treatment of subjects such as animals or humans.

The expression “pharmaceutically acceptable acid addition salt” as used herein means any non-toxic organic or inorganic salt of any compound of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluenesulfonic and methanesulfonic acids. Either the mono or di-acid salts can be formed, and such salts may exist in either a hydrated, solvated or substantially anhydrous form. In general, the acid addition salts of the compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the present disclosure, for laboratory use, or for subsequent conversion to a pharmaceutically acceptable acid addition salt.

The expression “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where CO₂H is a functional group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds of the disclosure, for laboratory use, or for subsequent conversion to a pharmaceutically acceptable acid addition salt.

The formation of a desired compound salt is achieved using standard techniques. For example, the neutral compound is treated with an acid or base in a suitable solvent and the formed salt is isolated by filtration, extraction or any other suitable method.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals such as a mouse, a rat, a dog and a human.

The terms “suitable” and “appropriate” mean that the selection of the particular group or conditions would depend on the specific synthetic manipulation to be performed and the identity of the molecule but the selection would be well within the skill of a person trained in the art. All process steps described herein are to be conducted under conditions suitable to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The expression a “therapeutically effective amount”, “effective amount” or a “sufficient amount” of a compound or composition of the present disclosure is a quantity sufficient to, when administered to the subject, including a mammal, for example a human, effect beneficial or desired results, including clinical results, and, as such, a “therapeutically effective amount” or an “effective amount” depends upon the context in which it is being applied. For example, in the context of treating cancer, it is an amount of the compound or composition sufficient to achieve such treatment of the cancer as compared to the response obtained without administration of the compound or composition. The amount of a given compound or composition of the present disclosure that will correspond to an effective amount will vary depending upon various factors, such as the given drug or compound, the pharmaceutical formulation, the route of administration, the type of disease or disorder, the identity of the subject or host being treated, and the like, but can nevertheless be routinely determined by one skilled in the art. Also, as used herein, a “therapeutically effective amount” or “effective amount” of a compound or composition of the present disclosure is an amount which inhibits, suppresses or reduces a cancer (e.g., as determined by clinical symptoms or the amount of cancerous cells) in a subject as compared to a control.

As used herein, and as well understood in the art, “treatment” or “treating” is an approach for obtaining beneficial or desired results, including clinical results. Beneficial or desired clinical results can include, but are not limited to, inhibition of inflammation or decrease of inflammation in a cell by at least about 10%, at least about 20%, at least about 30%, at least about 40%, at least about 50%, at least about 60%, at least about 70%, at least about 80% or at least about 90% greater than an untreated control cell. “Treatment” also means alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “tolerability” or “tolerated” as used herein means a degree to which a therapeutic agent may be endured or accepted by a subject treated with the therapeutic agent. For example, tolerability may be assessed by measuring different parameters such as (i) maintenance or absence of weight loss, (ii) duration of treatment withstood and (iii) decrease or absence of side effects. For example, it is well established that a therapeutic agent is tolerated by a subject when there is no weight loss observed during treatment using such a therapeutic agent. For example, the conjugates of the present disclosure (comprising at least one therapeutic agent) can increase the tolerability of a given therapeutic agent since the conjugate is being more selective to receptors than the therapeutic agent taken alone.

The term “administered” or “administering” as used herein means administration of a therapeutically effective amount of a compound or composition of the application to a cell either in vitro (e.g. a cell culture) or in vivo (e.g. in a subject).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In compositions comprising an “additional” or “second” component, the second component as used herein is chemically different from the other components or first component. A “third” component is different from the other, first, and second components, and further enumerated or “additional” components are similarly different.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

A platform allowing the transport of therapeutic agents into cancer cells for new therapies directed against primary and secondary tumours was previously developed. This approach utilizes peptide compounds derived from bacterial proteins or from ligands of receptors expressed in cancer cells (ex. sortilins/syndecans). In the present disclosure, the conjugation of therapeutic to one of these peptide compounds for use in treating inflammation is described. For example, phytochemicals, for example Curcumin, can be conjugated to the peptide compounds.

Disclosed herein are peptide compounds as well as conjugate compounds comprising at least one therapeutic agent connected to a peptide compound for use in treating inflammation.

Accordingly, a first aspect is a peptide compound having at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid
and wherein at least one protecting group and/or at least one labelling agent is optionally connected to said peptide at an N- and/or C-terminal end,
for use in treating inflammation.

For example, the peptide compound is a peptide compound that comprises:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

For example, the peptide compound is a peptide compound that consists essentially of:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

For example, the peptide compound is a peptide compound that consists of:

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
or
(XIII)
(SEQ ID NO: 13)
YKSLRRKAPRWDAYLRDPALRPLL.

According to another aspect, there is provided a peptide compound that comprises a compound chosen from compounds of formula (I), formula (II), formula (III), formula (IV), formula (V), formula (VI), formula (VII), formula (VIII), formula (IX), formula (X), formula (XI), formula (XII) and formula (XIII):

(I)
(SEQ ID NO: 1)
X₁X₂X₃X₄X₅GVX₆AKAGVX₇NX₈FKSESY
(II)
(SEQ ID NO: 2)
(X₉)ₙGVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL

wherein
   X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
   X₁₆, X₁₇, X₂₀ and X₂₁ are independently chosen from Q, P, Y, I and L;
   n is 0, 1, 2, 3, 4 or 5;
   when X₉ is present more than once, each of said X₉ is independently chosen from any amino acid;
   when X₁₉ is present more than once, each of said X₉ is independently chosen from any amino acid
   and wherein at least one protecting group and/or at least one labelling agent is optionally connected to said peptide at an N- and/or C-terminal end,
   for use in treating inflammation.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound chosen from peptide compounds of formula (I), formula (II), formula (III), formula (IV), formula (V), formula (VI), formula (VII), formula (VIII), formula (IX), formula (X), formula (XI), formula (XII) and formula (XIII).

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (I) or SEQ ID NO: 1.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (II) or SEQ ID NO: 2.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (III) or SEQ ID NO: 3.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IV) or SEQ ID NO: 4.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (V) or SEQ ID NO: 5.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VI) or SEQ ID NO: 6.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VII) or SEQ ID NO: 7.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (VIII) or SEQ ID NO: 8.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (IX) or SEQ ID NO: 9.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (X) or SEQ ID NO: 10.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XI) or SEQ ID NO: 11.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XII) or SEQ ID NO: 12.

For example, the peptide compound has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to a peptide compound represented by formula (XIII) or SEQ ID NO: 13.

In one embodiment, n is 0. In one embodiment, n is 1. In one embodiment, n is 2. In one embodiment, n is 3. In one embodiment, n is 4. In one embodiment, n is 5.

In an embodiment, the peptide compound is represented by formula (I) or formula (II).

In one embodiment, the peptide compound is represented by formula (I) or SEQ ID NO: 1.

In one embodiment, the peptide compound is represented by formula (II) or SEQ ID NO: 2.

In an embodiment, the peptide compound is represented by formula (V), formula (VI), formula (VII), formula (VIII), formula (IX) or formula (X).

In one embodiment, the peptide compound is represented by formula (V).

In one embodiment, the peptide compound is represented by formula (VI).

In one embodiment, the peptide compound is represented by formula (VII).

In one embodiment, the peptide compound is represented by formula (VIII).

In one embodiment, the peptide compound is represented by formula (IX).

In one embodiment, the peptide compound is represented by formula (X).

In one embodiment, the peptide compound is represented by formula (III) or formula (IV).

In one embodiment, the peptide compound is represented by formula (III).

In one embodiment, the peptide compound is represented by formula (IV).

In one embodiment, the peptide compound is represented by formula (XI), formula (XII) or formula (XIII).

In one embodiment, the peptide compound is represented by formula (XI).

In one embodiment, the peptide compound is represented by formula (XII).

In one embodiment, the peptide compound is represented by formula (XIII).

In one embodiment, the peptide is represented by the amino acid sequence of SEQ ID NO: 1. In one embodiment, the peptide is represented by the amino acid sequence of SEQ ID NO: 2. In one embodiment, the peptide is represented by the amino acid sequence of SEQ ID NO: 3. In one embodiment, the peptide is represented by the amino acid sequence of SEQ ID NO: 4. In one embodiment, the peptide is represented by the amino acid sequence of SEQ ID NO: 5. In one embodiment, the peptide is represented by the amino acid sequence of SEQ ID NO: 6. In one embodiment, the peptide is represented by the amino acid sequence of SEQ ID NO: 7. In one embodiment, the peptide is represented by the amino acid sequence of SEQ ID NO: 8. In one embodiment, the peptide is represented by the amino acid sequence of SEQ ID NO: 9. In one embodiment, the peptide is represented by the amino acid sequence of SEQ ID NO: 10. In one embodiment, the peptide is represented by the amino acid sequence of SEQ ID NO: 11. In one embodiment, the peptide is represented by the amino acid sequence of SEQ ID NO: 12. In one embodiment, the peptide is represented by the amino acid sequence of SEQ ID NO: 13.

In one embodiment, at least one protecting group is connected to said peptide at an N- and/or C-terminal end.

In one embodiment, a succinyl group is connected to the peptide compound. For example, the peptide compound has the sequence of Succinyl-IKLSGGVQAKAGVINMFKSESY, corresponding to SEQ ID NO: 6 and having a succinyl group attached thereto at the N-terminal end.

In one embodiment, an acetyl group is connected to the peptide compound. For example, the peptide compound has the sequence of Acetyl-GVRAKAGVRNMFKSESY (SEQ ID NO: 14). For example, the peptide compound has the sequence of Acetyl-GVRAKAGVRN(Nle)FKSESY (SEQ ID NO: 15). For example, the peptide compound has the sequence of Acetyl-YKSLRRKAPRWDAPLRDPALRQLL (SEQ ID NO: 16). For example, the peptide compound has the sequence of Acetyl-YKSLRRKAPRWDAYLRDPALRQLL (SEQ ID NO: 17). For example, the peptide compound has the sequence of Acetyl-YKSLRRKAPRWDAYLRDPALRPLL (SEQ ID NO: 18).

In one embodiment, at least one labelling agent is connected to said peptide at an N- and/or C-terminal end.

The person skilled in the art will understand that commonly used labelling agents can be used. For example, the labelling agent is a vitamin. For example, the labelling agent is biotin. For example, the labelling agent is used as a fluorescent probe and/or as an imaging agent.

In one embodiment, the peptide compound is biotinylated. For example, the peptide compound has the sequence of IKLSGGVQAKAGVINMFKSESYK(Biotin), corresponding to SEQ ID NO: 7 and having a biotin molecule attached thereto at the C-terminal end.

For example, the peptide compound is represented by Formula (XXXVI):

(XXXVI)
Succinyl-IKLSGGVQAKAGVINMFKSESY

that comprises the peptide compound having SEQ ID NO: 6 wherein a succinyl group is attached at the N-terminal end.

In one embodiment, X₁₆ is independently chosen from Q, P, Y, I and L.

For example, X₁₆ is Q.

For example, X₁₆ is P.

For example, X₁₆ is Y.

For example, X₁₆ is I.

In one embodiment, X₁₇ is independently chosen from Q, P, Y, I and L.

For example, X₁₇ is Q.

For example, X₁₇ is P.

For example, X₁₇ is Y.

For example, X₁₇ is I.

In one embodiment, X₂₀ is independently chosen from Q, P, Y, I and L.

For example, X₂₀ is Q.

For example, X₂₀ is P.

For example, X₂₀ is Y.

For example, X₂₀ is I.

In one embodiment, X₂₁ is independently chosen from Q, P, Y, I and L.

For example, X₂₁ is Q.

For example, X₂₁ is P.

For example, X₂₁ is Y.

For example, X₂₁ is I.

In one embodiment, the peptide compound is chosen from:

(SEQ ID NO: 1)
X₁X₂X₃X₄X₅GVX₆AKAGVX₇NX₈FKSESY;
(SEQ ID NO: 2)
(X₉)ₙGVX₁₀AKAGVX₁₁NX₁₂FKSESY;
(SEQ ID NO: 3)
YKX₁₃LRRX₁₄APRWDX₁₅PLRDPALRX₁₆X₁₇L;
(SEQ ID NO: 4)
YKX₁₈LRR(X₁₉)ₙPLRDPALRX₂₀X₂₁L;
(SEQ ID NO: 5)
IKLSGGVQAKAGVINMDKSESM;
Succinyl-IKLSGGVQAKAGVINMFKSESY (that comprises
SEQ ID NO: 6 wherein a succinyl group is attached
thereto at the N-terminal end);
IKLSGGVQAKAGVINMFKSESYK(Biotin) (that comprises
SEQ ID NO: 7 wherein a biotin molecule is attached
thereto at the C-terminal end);
(SEQ ID NO: 8)
GVQAKAGVINMFKSESY;
(SEQ ID NO: 14)
Acetyl-GVRAKAGVRNMFKSESY;
(SEQ ID NO: 15)
Acetyl-GVRAKAGVRN(Nle)FKSESY;
(SEQ ID NO: 16)
Acetyl-YKSLRRKAPRWDAPLRDPALRQLL;
(SEQ ID NO: 17)
Acetyl-YKSLRRKAPRWDAYLRDPALRQLL;
and
(SEQ ID NO: 18)
Acetyl-YKSLRRKAPRWDAYLRDPALRPLL.

In one embodiment, the peptide compounds can be modified at the C- and/or N-terminal by the addition of one or more amino acid residue in order to obtain or increase preferential binding sites at the peptide terminal end. For example, the amino acid can be cysteine. For example, the amino acid can be lysine.

The peptide compounds described herein can be connected, linked, mixed or conjugated to small molecules, peptides, proteins, oligonucleotides, diagnostic agents, imaging or radionuclide agents, large molecules such as monoclonal antibodies, therapeutic agents such phytochemicals or to drug delivery systems including nanoparticles, liposomes, nanotubes, graphene particles loaded with a therapeutic agent, imaging agent, gene, siRNA. The resulting conjugate compounds can be used as mono- or combined therapies for example for treating inflammation.

Accordingly, another aspect disclosed herein is a conjugate compound having the formula of A-(B)ₙ,
wherein
   n is 1, 2, 3 or 4;
   A is a peptide compound as defined herein, wherein said peptide is optionally protected by a protecting group; and
   B is at least one therapeutic agent, wherein B is connected to A,
   for use in treating inflammation.

Yet another aspect disclosed herein is a conjugate compound having the formula of A-(B)ₙ,

wherein
n is 1, 2, 3 or 4;
A is a peptide compound as defined herein; and
B is at least one therapeutic agent, wherein B is connected to A at a free amine of a lysine residue of said peptide compound, optionally via a linker, or at an N-terminal position of said peptide compound, optionally via a linker,
for use in treating cancer.

In an embodiment, B is connected to A via a linker, optionally a cleavable linker.

For example, the at least one therapeutic agent is an anti-inflammatory agent.

For example, the anti-inflammatory agent is a phytochemical, a non-steroidal anti-inflammatory drug, a steroidal anti-inflammatory drug, an antileukotrine agent, a biologic agent or an immune-selective anti-inflammatory derivative (ImSAID).

For example, the anti-inflammatory agent is a phytochemical chosen from curcumin, omega-3, white willow bark, green tea, catechins, pycnogenol, Boswellia serrata resin, resveratrol, Uncaria tomentosa, capsaicin, anthocyanins/anthocyanidins, flavanoids, olive oil compounds, chlorogenic acid and sulfopharaphane.

For example, the anti-inflammatory agent is a non-steroidal anti-inflammatory drug chosen from Aspirin (Anacin, Ascriptin, Bayer, Bufferin, Ecotrin, Excedrin), Choline and magnesium salicylates (CMT, Tricosal, Trilisate), Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Bayer Select, Doan's Pills, Magan, Mobidin, Mobogesic), Meclofenamate sodium (Meclomen), Mefenamic acid (Ponstel), Meloxicam (Mobic), Nabumetone (Relafen), Naproxen (Naprosyn, Naprelan*), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril), and Tolmetin sodium (Tolectin).

For example, the anti-inflammatory agent is a steroidal anti-inflammatory drug chosen from Hydrocortisone type drugs, for example Hydrocortisone, methylprednisolone, prednisolone, prednisone, and triamcinolone (short- to medium-acting glucocorticoid), Acetonides for example Amcinonide, budesonide, desonide, fluocinolone acetonide, fluocinonide, halcinonide, and triamcinolone acetonide, Betamethasone type drugs, for example Beclometasone, betamethasone, dexamethasone, fluocortolone, halometasone, and mometasone, esters, for example: Halogenated esters (less labile) such as Alclometasone dipropionate, betamethasone dipropionate, betamethasone valerate, clobetasol propionate, clobetasone butyrate, fluprednidene acetate, and mometasone furoate, and Labile prodrug esters, such as Ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

Antileukotrines are anti-inflammatory agents which function as leukotriene-related enzyme inhibitors (arachidonate 5-lipoxygenase) or leukotriene receptor antagonists (cysteinyl leukotriene receptors) and consequently oppose the function of these inflammatory mediators. For example, the anti-inflammatory agent is a antileukotrine agent chosen from Leukotriene receptor antagonists, such as montelukast, zafirlukast, and pranlukast, and 5-lipoxygenase inhibitors, such as zileuton and Hypericum perforatum.

For example, the anti-inflammatory agent is a biologic agent chosen from Rituximab, Abatacept, Tocilizumab, Etanercept, Adalimumab, Infliximab, Ankinra.

ImSAIDs are a new category of anti-inflammatory agents that are unrelated to steroid hormones or non-steroidal anti-inflammatory agents. One ImSAID in particular is a SGP-T derivative which is a three-amino acid sequence shown to be a potent anti-inflammatory molecule with systemic effects. This three-amino acid peptide that is phenylalanine-glutamine-glycine (FEG) and its D-isomeric form (feG) have become the foundation for the ImSAID agents. For example, the anti-inflammatory agent is an ImSAID that is a SGP-T derivative.

In an embodiment, the phytochemical is curcumin.

In an embodiment, the conjugate compound is chosen from:

Acetyl-GVRAK(curcumin)AGVRN(Nle)FK(curcumin)SESY Formula (XIV)

that comprises the peptide compound having SEQ ID NO: 15 wherein each lysine residue has a curcumin molecule connected thereto, and

Acetyl-YK(curcumin)SLRRK(curcumin)APRWDAPLRDPALRQLL Formula (XV)

that comprises the peptide compound having SEQ ID NO: 16 wherein each lysine residue has a curcumin molecule connected thereto.

For example, the conjugate compound is represented by formula (XIV).

For example, the conjugate compound is represented by formula (XV).

In an embodiment, B, the at least one therapeutic agent, is connected to A, the peptide compound, at said free amine of said lysine residue of said peptide compound, via a linker.

In an embodiment, B, the at least one therapeutic agent, is connected to A, the peptide compound, at said N-terminal position of said peptide compound, via a linker.

In an embodiment, the linker is chosen from succinic acid and dimethyl glutaric acid linker.

For example, the linker is a cleavable linker.

For example, the linker is a non-cleavable linker.

For example, the conjugate compound can comprise a cleavable linker connected the at least one therapeutic agent to the peptide compound. For example, the at least one therapeutic agent can be released from the peptide compound by the action of esterases on the ester bond.

For example, a therapeutic agent can be conjugated to the peptide compound on free amines available on the peptide, at the lysine or amino-terminal, by forming a bond such as a peptide bond.

In an embodiment, the conjugate compound comprises 1 molecule of the therapeutic agent connected to the peptide compound.

In an embodiment, the conjugate compound comprises 2 molecules of the therapeutic agent connected to the peptide compound.

In an embodiment, the conjugate compound comprises 3 molecules of the therapeutic agent connected to the peptide compound.

In an embodiment, the conjugate compound comprises 4 molecules of the therapeutic agent connected to the peptide compound.

For example, the inflammation is TNF-α-induced inflammation.

For example, the treating inflammation comprises inhibiting TNF-α-induced COX-2 expression in cells.

For example, the treating inflammation comprises decreasing TNF-α-induced COX-2 expression in cells expressing Sortilin by at least about 20%, at least about 30%, at least about 40%, at least about 50%, at least about 60%, at least about 70%, at least about 80% or at least about 90% greater than untreated cells expressing Sortilin.

For example, the treating inflammation comprises decreasing TNF-α-induced COX-2 expression in cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

For example, the treating inflammation comprises inhibiting TNF-α-induced IκB phosphorylation in cells expressing Sortilin.

For example, the treating inflammation comprises decreasing TNF-α-induced IκB phosphorylation in cells expressing Sortilin by at least about 20%, at least about 30%, at least about 40%, at least about 50%, at least about 60%, at least about 70%, at least about 80% or at least about 90% greater than untreated cells expressing Sortilin.

For example, the treating inflammation comprises decreasing TNF-α-induced IκB phosphorylation in cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

For example, the inflammation can be caused by an inflammatory disease.

For example, the inflammatory disease can be chosen from rheumatoid arthritis, ankylosing spondylitis, inflammatory bowel disease, psoriasis, cancer, pain, osteoarthritis, inflammatory bowel disease, Crohn's disease, colitis, dermatitis, diverticulitis, fibromyalgia, hepatitis, systemic lupus erythematous, acne vulgaris, chronic prostatitis, ulcerative colitis, ankylosing spondylitis, diseases of the central nervous system, for example autoimmune encephalomyelitis, Alzheimer's disease, Parkinson's disease and traumatic brain injury, cardiovascular disease, for example atherosclerosis, inflammatory lung disease, for example chronic bronchitis, chronic obstructive pulmonary disease, acute respiratory distress syndrome and asthma, renal inflammatory disease, for example ischaemic renal injury, renal transplant rejection and glomerulonephritis, reperfusion injury, sarcoidosis and pelvic inflammation.

For example, the cells expressing Sortilin are immune cells, optionally macrophages, CD4+, CD8+, B220+, bone marrow-derived cells basophils, eosinophils and cytotoxic T lymphocytes, Natural Killer (NK) cells, T helper type 1 (Th1) cells.

For example, the cells expressing Sortilin are cancer cells, optionally ovarian cancer cells, endometrial cancer cells, breast cancer cells, prostate cancer cells, colorectal cancer cells, lung cancer cells, pancreas cancer cells, skin cancer cells, brain (gliomas) cancer cells, urothelial cancer cells, carcinoid cancer cells, renal cancer cells, testis cancer cells, pituitary cancer cells and blood cancer cells such as bone marrow cancer cells, diffuse large B cell lymphoma cancer cells, myeloma cancer cells or chronic B cell leukemia cancer cells.

Conjugate compounds herein disclosed can also be used to transport therapeutic agents into the cell as they are not a substrate of efflux pumps such as the P-glycoprotein membrane transporter pump which pumps out other therapeutic agents from multi resistant drug cells.

In a further aspect, there is provided a process for preparing the conjugate compound herein disclosed, the process comprising:
reacting a linker together with said therapeutic agent so as to obtain an intermediate;
optionally purifying said intermediate;
reacting said intermediate together with said peptide compound so as to obtain said conjugate compound; and
optionally purifying said conjugate compound;
   wherein the therapeutic agent is connected to the peptide compound at a free amine of a lysine residue or an N-terminal; and wherein the peptide compound comprises 1, 2, 3 or 4 therapeutic agent molecules connected thereto.

For example, the peptide compound comprises 1 therapeutic agent molecule connected thereto. For example, the peptide compound comprises 2 therapeutic agent molecules connected thereto. For example, the peptide compound comprises 3 therapeutic agent molecules connected thereto. For example, the peptide compound comprises 4 therapeutic agent molecules connected thereto.

For example, the linker is succinic acid.

For example, the linker is a dimethyl glutaric acid linker.

In an embodiment, the peptide compound is protected at said N-terminal prior to reacting with said intermediate.

For example, a protecting group such as FMOC can be added as a protecting group to a free amine on the therapeutic agent prior to incorporation with a linker. After its synthesis, the conjugate compound can undergo deprotection from the protecting group. For example, the conjugate compound comprising the protecting agent FMOC can be deprotected using piperidin. The person skilled in the art would readily understand that other known chemical reagents may be used for deprotection of conjugate compounds.

For example, the N-terminal of the therapeutic agent and/or the peptide compound can be capped by its acetylation, thereby providing a non-reversible protecting group at the N-terminal.

In an embodiment, the intermediate is activated prior to reacting with said peptide compound.

For example, the intermediate is activated prior to reacting with said compound with a coupling agent, optionally chosen from N,N,N′,N′-Tetramethyl-O-(benzotriazol-1-yl)uronium tetrafluoroborate (TBTU), (2-(1H-benzotriazol-1-yl)-1,1,3,3-tetramethyluronium hexafluorophosphate) (HBTU), and (1-[Bis(dimethylamino)methylene]-1H-1,2,3-triazolo[4,5-b]pyridinium 3-oxid hexafluorophosphate) (HATU).

For example, the intermediate comprising a therapeutic agent connected to a linker can be activated with TBTU, a peptide coupling reagent, prior to conjugation with the peptide compound.

In one embodiment, the conjugate compound is purified following its synthesis.

Compounds disclosed herein may also be used in the context of fusion proteins. For example, a fusion protein can be engineered by fusing a compound herein disclosed, for example a peptide compound, to one or more proteins, or parts thereof such as functional domains. Fusion proteins can be engineered for example by recombinant DNA technology and expressed using a protein expression system such as a bacterial or mammalian protein expression system. In some embodiments, peptide linkers are added between proteins. In other embodiment, the fusion proteins do not comprise linkers connecting the proteins.

Commonly used protein expression systems include those derived from bacteria, yeast, baculovirus/insect, plants and mammalian cells and more recently filamentous fungi such as the Myceliophthora thermophile.

An aspect herein disclosed is a liposome, graphene, nanotube or nanoparticle comprising at least one compound disclosed herein for use in treating inflammation.

Another aspect is a liposome, graphene, nanotube or nanoparticle coated with at least one compound disclosed herein for use in treating inflammation.

Another aspect is a liposome, graphene, nanotube or nanoparticle loaded with at least one therapeutic agent, gene or siRNA; and the liposome or nanoparticle is coated with at least one compound herein defined, for use in treating inflammation. For example, the at least one compound can be connected to the surface of the liposome or nanoparticle.

Different embodiments of liposomes, nanotubes, graphene or nanoparticles can be envisaged by the person skilled in the art. For example the liposome or nanoparticle can comprise at least one peptide compound herein disclosed coated on the surface of the liposome or nanoparticle and a therapeutic agent, for example an anticancer agent, within the liposome or nanoparticle. For example, the liposome or nanoparticle can comprise at least one conjugate compound herein disclosed coated on the surface of the liposome or nanoparticle and a therapeutic agent, for example an anticancer agent, within the liposome or nanoparticle. In addition, in some embodiments, the compound herein described can be associated, linked, or connected to one or more other compounds to form a multimer such as a dimer, a trimer or a tetramer, as well as branched peptides. Such compounds can be connected together, for example via a covalent bond, an atom or a linker. For example, the multimer comprises more than one peptide compound and/or more than one conjugate compound. Methods for making multimeric (e.g. dimeric, trimeric) forms of compounds are described in U.S. Pat. No. 9,161,988 which is incorporated herein by reference in its entirety.

Other aspects of the present disclosure generally include methods of treating inflammation comprising administering a therapeutically effective amount of at least one compound herein disclosed to a subject in need thereof and/or contacting cells expressing Sortilin with at least one compound herein disclosed. Other aspects include uses of the compounds described herein for treating inflammation as well as in the manufacture of a medicament for treatment inflammation.

In an aspect, there is provided a method of treating inflammation comprising administering to a subject in need thereof a therapeutically effective amount of at least one compound as defined herein.

In another aspect, there is provided a method of treating TNF-α-induced inflammation, comprising administering to a subject in need thereof a therapeutically effective amount of at least one compound as defined herein.

In another aspect, there is provided a method of treating inflammation in cells expressing Sortilin, comprising contacting said cells with at least one compound as defined herein.

In another aspect, there is provided a method of inhibiting TNF-α-induced COX-2 expression in cells expressing Sortilin, comprising contacting said cells with at least one compound as defined herein.

In another aspect, there is provided a method of decreasing TNF-α-induced COX-2 expression in cells expressing Sortilin, comprising contacting said cells with at least one compound as defined herein, wherein the TNF-α-induced COX-2 expression is decreased by at least about 20%, at least about 30%, at least about 40%, at least about 50%, at least about 60%, at least about 70%, at least about 80% or at least about 90% greater than untreated cells expressing Sortilin.

In another aspect, there is provided a method of decreasing TNF-α-induced COX-2 expression in cells expressing Sortilin, comprising contacting said cells with at least one compound as defined herein, wherein the TNF-α-induced COX-2 expression is decreased by at least 1.2, at least 1.4, at least 1.6, at least 1.8, at least 2.0, at least 2.2 or at least 2.4 fold greater than cells expressing Sortilin treated with the at least one therapeutic agent.

In another aspect, there is provided a method of inhibiting TNF-α-induced IκB phosphorylation in cells expressing Sortilin, comprising contacting said cells with at least one compound as defined herein.

In another aspect, there is provided a method of decreasing TNF-α-induced IκB phosphorylation in cells expressing Sortilin, comprising contacting said cells with at least one compound as defined herein, wherein the TNF-α-induced IκB phosphorylation is decreased by at least about 20%, at least about 30%, at least about 40%, at least about 50%, at least about 60%, at least about 70%, at least about 80% or at least about 90% greater than untreated cells expressing Sortilin.

In another aspect, there is provided a method of decreasing TNF-α-induced IκB phosphorylation in cells expressing Sortilin, comprising contacting said cells with at least one compound as defined herein, wherein the TNF-α-induced IκB phosphorylation is decreased by at least 1.2, at least 1.4, at least 1.6, at least 1.8, at least 2.0, at least 2.2 or at least 2.4 fold greater than cells expressing Sortilin treated with the at least one therapeutic agent.

In another aspect, there is provided a method of increasing stability and/or bioavailability of a therapeutic agent, comprising:
obtaining the conjugate compound disclosed herein, wherein said conjugate compound comprises said therapeutic agent, and
administering a therapeutically effective amount of said conjugate compound to a subject in need thereof.

In another aspect, there is provided a method of increasing stability and/or bioavailability of a therapeutic agent, comprising:
conjugating said therapeutic agent with the peptide compound as defined herein to obtain a conjugate compound, and
administering a therapeutically effective amount of said conjugate compound to a subject in need thereof.

The conjugate compounds herein disclosed may also provide greater tolerability compared to unconjugated therapeutic agents. For example, in the International application published as WO 2017/088058 and entitled PEPTIDE COMPOUNDS AND PEPTIDE CONJUGATES FOR THE TREATMENT OF CANCER THROUGH RECEPTOR-MEDIATED CHEMOTHERAPY, filed Nov. 24, 2016 (herein incorporated by reference in its entirety), it has been shown in that Katana-drug conjugates are better tolerated compared to unconjugated therapeutic agents at an equivalent dose due to specific receptor targeting. In particular, in vivo studies showed that treatment with a conjugate compound had little effect on the body weight of tested mice thus demonstrating tolerability of the conjugate compound.

For example, there is provided herein a method of increasing tolerability of a therapeutic agent, comprising:
conjugating the therapeutic agent with the peptide compound herein disclosed to obtain a conjugate compound, and
administering a therapeutically effective amount of the conjugate compound to a subject in need thereof.

For example, there is provided herein a method of increasing tolerability of a therapeutic agent, comprising:
obtaining a conjugate compound herein disclosed, wherein the conjugate compound comprises the therapeutic agent, and
administering a therapeutically effective amount of the conjugate compound to a subject in need thereof.

For example, there is provided a use of a conjugate compound herein disclosed, for increasing tolerability of a therapeutic agent.

In another aspect, there is provided a use of at least one compound as defined herein for treating inflammation.

In another aspect, there is provided a use of at least one compound as defined herein for treating TNF-α-induced inflammation.

In another aspect, there is provided a use of at least one compound as defined herein for treating an inflammatory disease.

In another aspect, there is provided a use of at least one compound as defined herein for treating a TNF-α-induced inflammatory disease.

In another aspect, there is provided a use of at least one compound as defined herein for treating an inflammatory disease involving sortilin expression.

In another aspect, there is provided a use of at least one compound as defined herein for inhibiting TNF-α-induced COX-2 expression in cells expressing Sortilin.

In another aspect, there is provided a use of at least one compound as defined herein for decreasing TNF-α-induced COX-2 expression in cells expressing Sortilin by at least about 20%, at least about 30%, at least about 40%, at least about 50%, at least about 60%, at least about 70%, at least about 80% or at least about 90% greater than untreated cells expressing Sortilin.

In another aspect, there is provided a use of at least one compound as defined herein for decreasing TNF-α-induced COX-2 expression in cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

In another aspect, there is provided a use of at least one compound as defined herein for inhibiting TNF-α-induced IκB phosphorylation in cells expressing Sortilin.

In another aspect, there is provided a use of at least one compound as defined herein for decreasing TNF-α-induced IκB phosphorylation in cells expressing Sortilin by at least about 20%, at least about 30%, at least about 40%, at least about 50%, at least about 60%, at least about 70%, at least about 80% or at least about 90% greater than untreated cells expressing Sortilin.

In another aspect, there is provided a use of at least one compound as defined herein for decreasing TNF-α-induced IκB phosphorylation in cells expressing Sortilin by at least 1.2, at least 1.4, at least 1.6, at least 1.8, at least 2.0, at least 2.2 or at least 2.4 fold greater than cells expressing Sortilin treated with the at least one therapeutic agent.

In another aspect, there is provided a use of a conjugate compound as defined herein for increasing stability and/or bioavailability of said at least one therapeutic agent.

In another aspect, there is provided a use of one compound as defined herein in the manufacture of a medicament for treating inflammation.

In another aspect, there is provided a use of one compound as defined herein in the manufacture of a medicament for treating TNF-α-induced inflammation.

In another aspect, there is provided a use of one compound as defined herein in the manufacture of a medicament for treating a TNF-α-induced inflammatory disease.

In another aspect, there is provided a use of one compound as defined herein in the manufacture of a medicament for treating an inflammatory disease involving sortilin expression.

In another aspect, there is provided a use of one compound as defined herein in the manufacture of a medicament for treating TNF-α-induced inflammation.

For example, the inflammation is caused by an inflammatory disease.

For example, the inflammatory disease is chosen from rheumatoid arthritis, ankylosing spondylitis, inflammatory bowel disease, psoriasis, cancer, pain, osteoarthritis, inflammatory bowel disease, Crohn's disease, colitis, dermatitis, diverticulitis, fibromyalgia, hepatitis, systemic lupus erythematous, acne vulgaris, chronic prostatitis, ulcerative colitis, ankylosing spondylitis, diseases of the central nervous system, for example autoimmune encephalomyelitis, Alzheimer's disease, Parkinson's disease and traumatic brain injury, cardiovascular disease, for example atherosclerosis, inflammatory lung disease, for example chronic bronchitis, chronic obstructive pulmonary disease, acute respiratory distress syndrome and asthma, renal inflammatory disease, for example ischaemic renal injury, renal transplant rejection and glomerulonephritis, reperfusion injury, sarcoidosis and pelvic inflammation.

For example, the at least one therapeutic compound comprised in the conjugate compound and/or used in the manufacture of a medicament to treat inflammation is an anti-inflammatory agent.

For example, the anti-inflammatory agent is a phytochemical, a non-steroidal anti-inflammatory drug, a steroidal anti-inflammatory drug, an antileukotrine agent, a biologic agent or an immune-selective anti-inflammatory derivative (ImSAID).

For example, the anti-inflammatory agent is a phytochemical chosen from curcumin, omega-3, white willow bark, green tea, catechins, pycnogenol, Boswellia serrata resin, resveratrol, Uncaria tomentosa, capsaicin, anthocyanins/anthocyanidins, flavanoids, olive oil compounds, chlorogenic acid and sulfopharaphane.

For example, the anti-inflammatory agent is a non-steroidal anti-inflammatory drug chosen from Aspirin (Anacin, Ascriptin, Bayer, Bufferin, Ecotrin, Excedrin), Choline and magnesium salicylates (CMT, Tricosal, Trilisate), Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Bayer Select, Doan's Pills, Magan, Mobidin, Mobogesic), Meclofenamate sodium (Meclomen), Mefenamic acid (Ponstel), Meloxicam (Mobic), Nabumetone (Relafen), Naproxen (Naprosyn, Naprelan*), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril), and Tolmetin sodium (Tolectin).

For example, the anti-inflammatory agent is a steroidal anti-inflammatory drug chosen from Hydrocortisone type drugs, for example Hydrocortisone, methylprednisolone, prednisolone, prednisone, and triamcinolone (short- to medium-acting glucocorticoid), Acetonides for example Amcinonide, budesonide, desonide, fluocinolone acetonide, fluocinonide, halcinonide, and triamcinolone acetonide, Betamethasone type drugs, for example Beclometasone, betamethasone, dexamethasone, fluocortolone, halometasone, and mometasone, esters, for example: Halogenated esters (less labile) such as Alclometasone dipropionate, betamethasone dipropionate, betamethasone valerate, clobetasol propionate, clobetasone butyrate, fluprednidene acetate, and mometasone furoate, and Labile prodrug esters, such as Ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

For example, the anti-inflammatory agent is a antileukotrine agent chosen from Leukotriene receptor antagonists, such as montelukast, zafirlukast, and pranlukast, and 5-lipoxygenase inhibitors, such as zileuton and Hypericum perforatum.

For example, the anti-inflammatory agent is a biologic agent chosen from Rituximab, Abatacept, Tocilizumab, Etanercept, Adalimumab, Infliximab, Ankinra.

For example, the anti-inflammatory agent is an ImSAID that is a SGP-T derivative.

Further embodiments of the present disclosure will now be described with reference to the following Examples. It should be appreciated that these Examples are for the purposes of illustrating embodiments of the present disclosure, and do not limit the scope of the disclosure.

### Examples

### Introduction

Curcumin (diferu-loylmethane), a naturally occurring polyphenol, is a phytochemical agent that is derived from turmeric (Curcuma longa L.). Clinical trials have demonstrated the efficacy and safety of curcumin supplementation in several human diseases (Sahebkar et al. 2016) such as osteoarthritis, metabolic syndrome, solid tumors, chronic obstructive pulmonary disease, anxiety and depression, rheumatoid arthritis psoriasis, pruritic skin disease and hypertriglyceridemia.

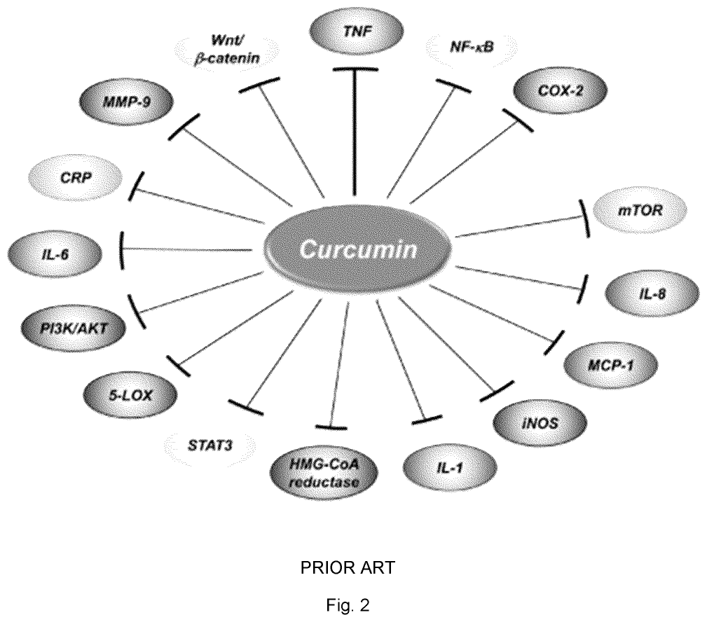

The underlying mechanism for curcumin pharmacological efficacy seems to occur through the modulation of numerous signaling molecules (FIG. 2). In light of the anti-inflammatory potential of Curcumin, this phytochemical has been conjugated with Katana peptides to better target Curcumin to cancer cells or immune cells expressing Sortilin. The anti-inflammatory potential of these Curcumin conjugates was then investigated. Results indicate that conjugation of Curcumin to Katana peptides increased its action against TNF-α-induced inflammatory pathways. Other anti-inflammatory drugs (Nonsteroidal and steroidal anti-inflammatory drugs) may gain from their conjugation to Katana peptides.

### Results

The chemical structures of 2 Curcumin-Katana peptide conjugates (KBC-106 and KBC-201) are described below. In these examples, Curcumin was conjugated using a cleavable linker to one peptide of each of the 2 Katana family peptides. KBC-201 was not described in WO 2017/088058. LC/MS analysis show a molecular weight of 3947.56 for KBC-201 and a molecular weight of 2909.24 for KBC-106

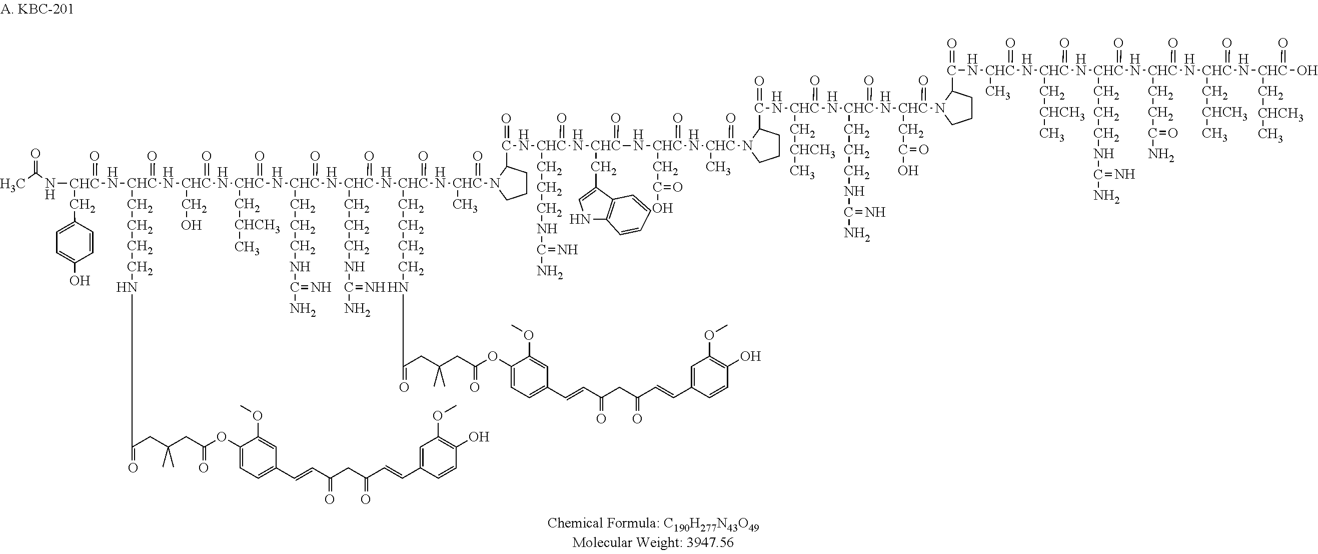

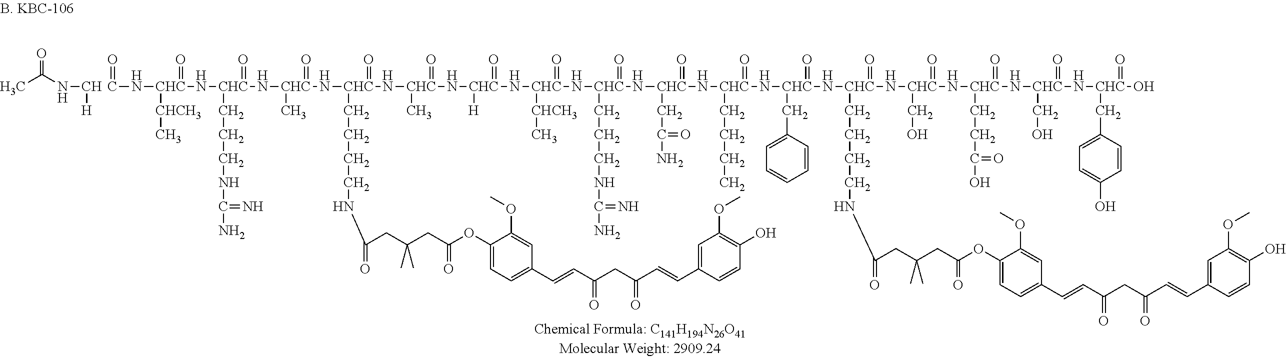

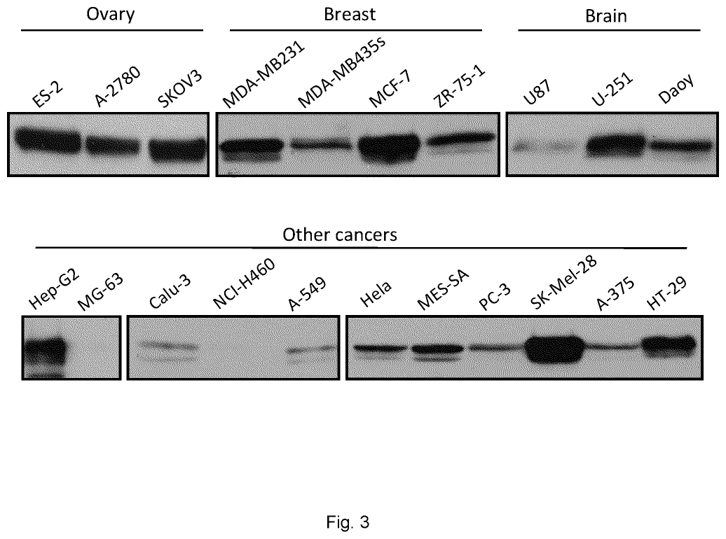

Sortilin expression was detected in various cancer cells by Western blotting (FIG. 3). The cancer cell lines tested are: human ovarian cancer cells: ES-2, SKOV3, A-2780; human breast cancer cells: MDA-MB231, MDA-MB435s, MCF-7, ZR-75-1; human brain cancer cells: U87, U-251, Daoy; and other human cancer cells: Hep-G2, MG-63, Calu-3, NCI-H460, A-549, Hela, MES-SA, PC-3, SK-Mel-28, A-375, HT-29. Results show high levels of Sortilin expression in many cancer cells including ovarian, breast, brain, melanoma and colorectal cancers.

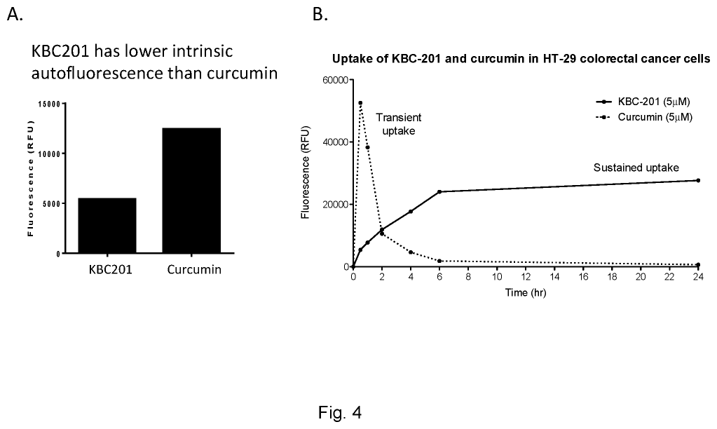

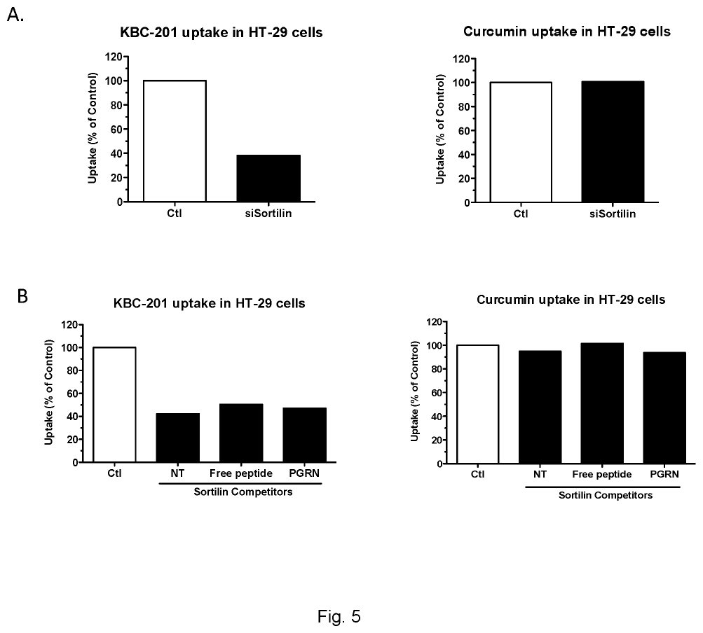

The uptake of Curcumin conjugate or free Curcumin was measured as a function of time in human HT-29 colon cancer cells (FIG. 4). FIG. 4A shows that the conjugation of Curcumin affects its intrinsic fluorescence. Indeed, the Curcumin conjugate (KBC-201) is less fluorescent by about 2-3 folds when compared to free Curcumin. Despite this lower intrinsic fluorescence, the uptake of KBC-201 was higher and sustained over time whereas transient and low intracellular accumulation was measured for the free Curcumin (FIG. 4B). Furthermore, when Sortilin expression in human HT-29 colon cancer cells was reduced using siRNA, the uptake of KBC-201 was strongly inhibited whereas that of the free Curcumin was unaffected (FIG. 5A). The uptake of both KBC-201 and free Curcumin was next measured in the presence of Sortilin ligands (FIG. 5B). Results demonstrate that the addition of free peptide and two Sortlilin ligands (Neurotensin and Progranulin) inhibited the uptake of the Curcumin conjugate (KBC-201). In contrast, free Curcumin uptake was unaffected by none of them indicating that Sortilin is involved in KBC-201 internalization. Taken together, data of the pharmacological inhibition using Sortilin ligands and the silencing of Sortlin expression confirm that the Curcumin conjugate is internalized via a Sortilin-dependent mechanism.

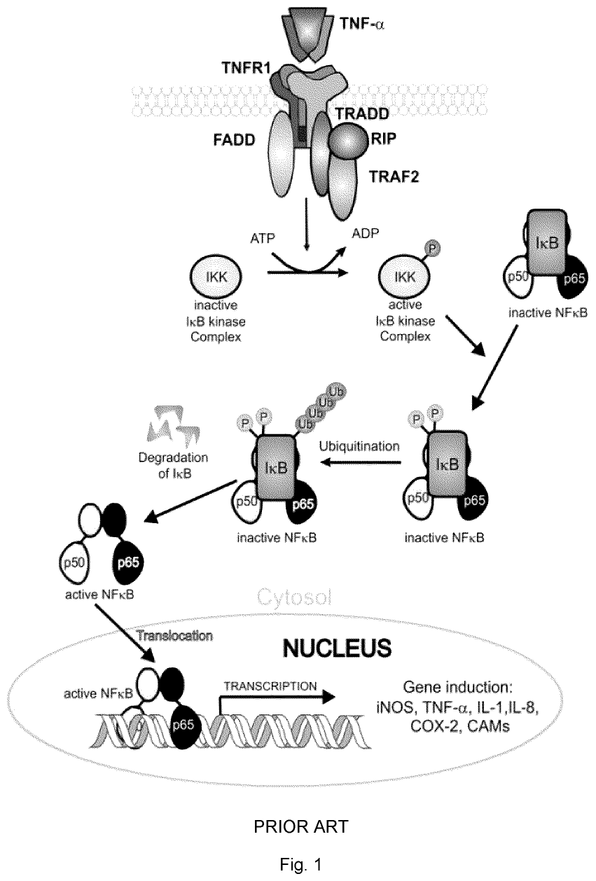

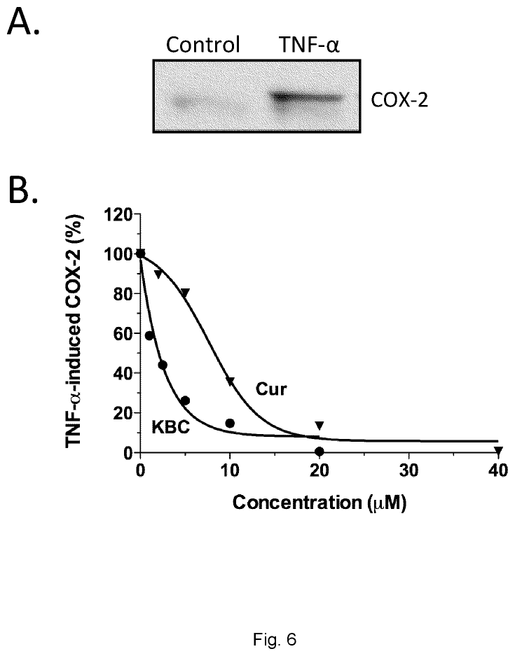

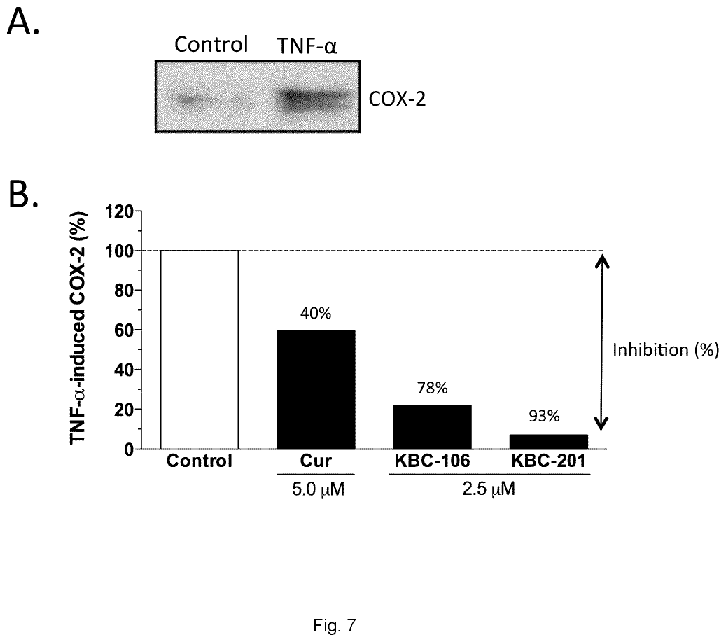

As indicated in FIG. 1 TNF-α induces different inflammatory pathways. In particular, the addition of TNF-α to human HT-29 cancer cells triggered the expression of COX-2 (FIG. 6A). Interestingly, the Curcumin conjugate (KBC-201) caused a stronger inhibition on the TNFα-induced COX-2 expression as compared to free Curcumin (FIG. 6B). In another experiment, the effect of two Curcumin conjugates (KBC-106, KBC-201) and free Curcumin on TNFα-induced COX-2 expression was evaluated (FIG. 7). Results indicate that both Curcumin conjugates were more potent than free Curcumin. KBC-201 showed the greatest inhibition of TNF-α-induced COX-2 expression.

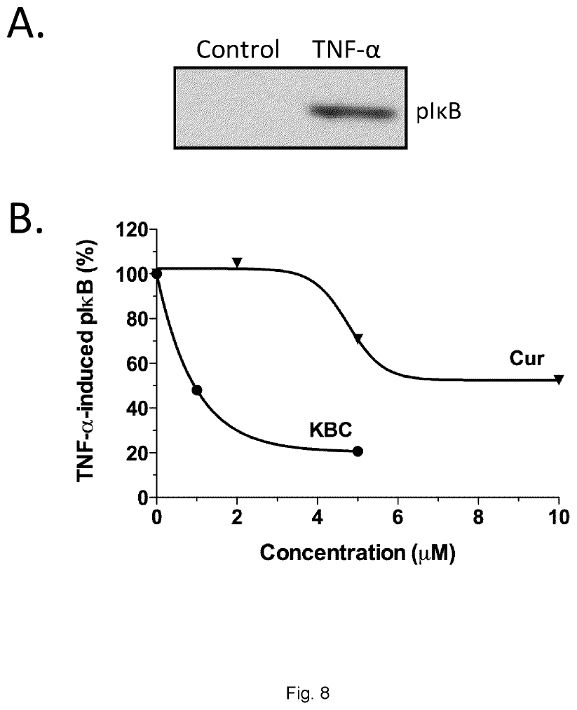

One of the inflammatory pathways induced by TNF-α leads to the phosphorylation of IkB (FIG. 1), a key protein in inflammation. As expected, the addition of TNF-α to human HT-29 colon cancer cells triggered the phosphorylation of IkB (pIkB) (FIG. 8). Similar to COX-2 expression, the addition of KBC-201 caused a stronger inhibition of TNF-α-induced IkB phosphorylation compared to free Curcumin.

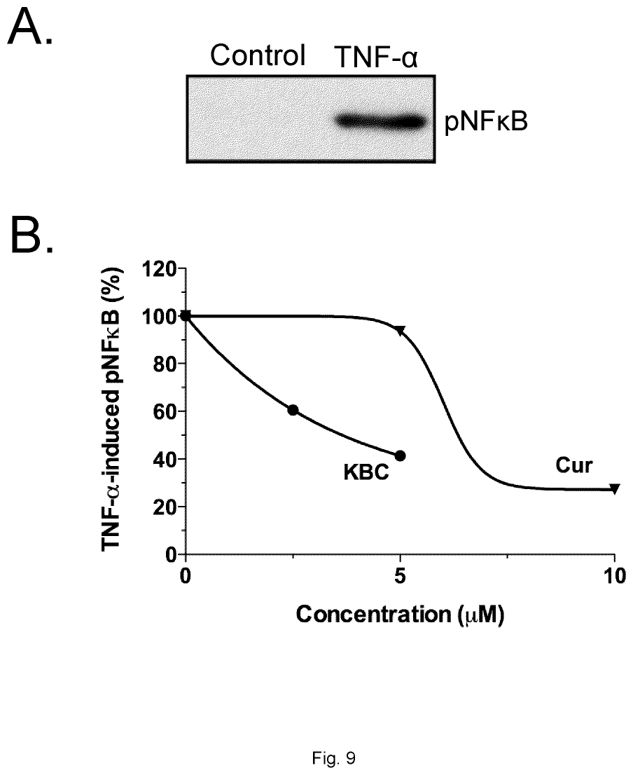

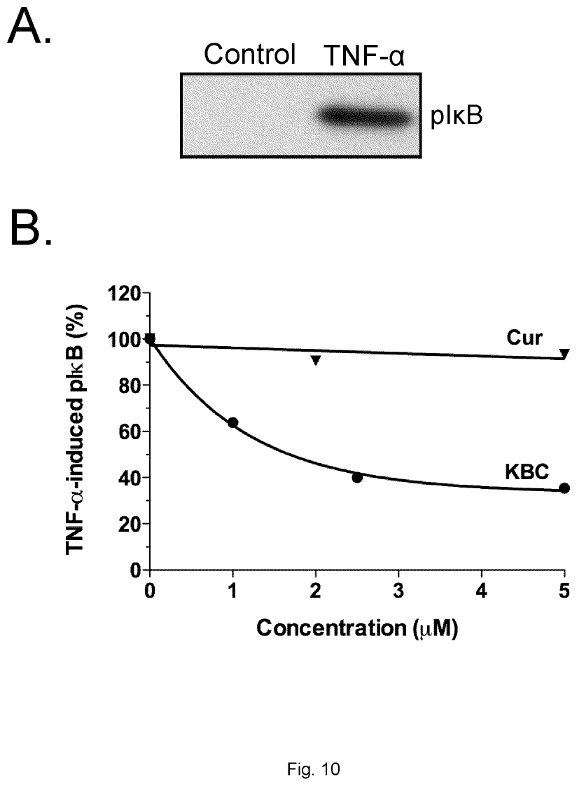

FIGS. 9 and 10 are other examples of the Curcumin conjugate (KBC-201)'s effect on the TNF-α-induced signaling pathways in two other cancer cell models. KBC-201 is more potent than free Curcumin to antagonize the phosphorylation of key pro-inflammatory proteins induced by TNF-α in the MDA-MB231 breast cancer cell model (FIG. 9) and in the SKOV3 ovarian cancer cell model (FIG. 10).

In addition to the inhibition potential on TNF-α-induced pro-inflammatory pathways, the stability of both Curcumin conjugates (KBC-106 and -201) was compared to that of free Curcumin at room temperature (FIG. 11). The absorbance of free Curcumin decreased more rapidly over time compared to both Curcumin conjugates indicating that the Curcumin conjugates are more stable. This suggests that the conjugation of Curcumin to Katana peptide(s) may increase the stability of this phytochemical compound. Since it has been reported that Curcumin has a poor stability or bioavailability the fact that both Conjugates are more stable may further increase their in vivo potency when compared to free Curcumin.

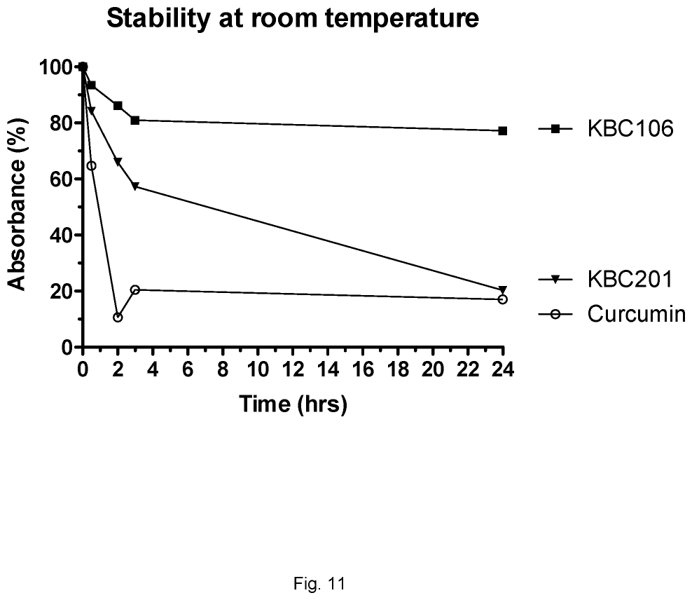

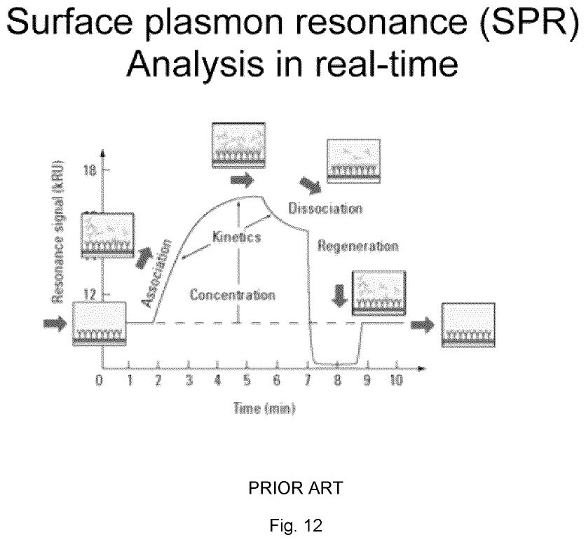

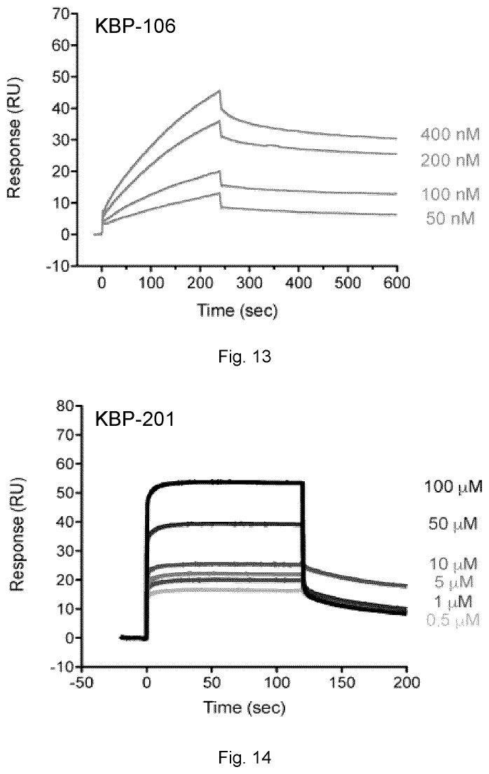

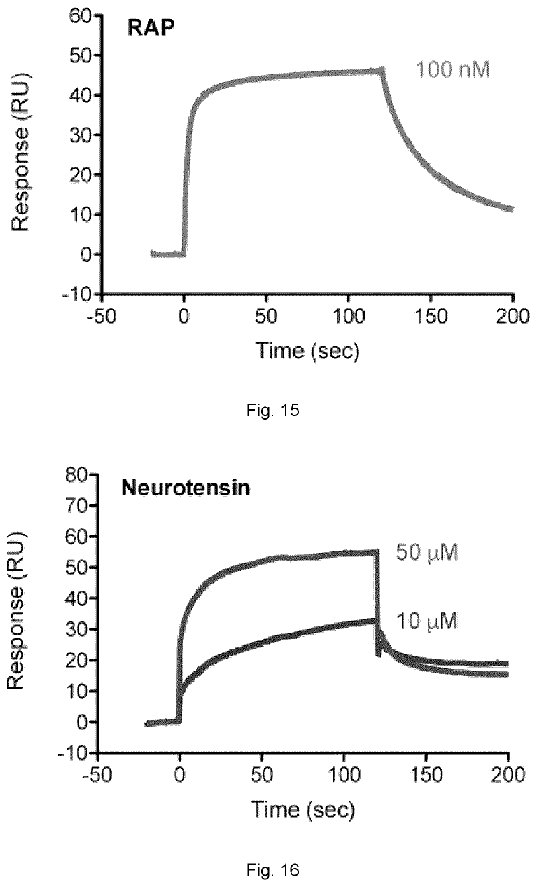

Further tests were made so as to investigate interaction of the peptide compounds of the present disclosure with Sortilin. Such tests were made using real-time surface plasmon resonance (Biacore) (see FIG. 12). Human recombinant Sortilin chimera protein from R&D Systems (#3154-ST) was immobilized on CM5 sensor chip with amine coupling standard manufacturer's procedures. After immobilization of recombinant Sortilin chimera protein, two peptide compounds of the present disclosure (KBP-106 in FIG. 13 and KBP-201 in FIG. 14) and Sortilin ligands (receptor-associated protein (RAP) in FIG. 15 and Neurotensin in FIG. 16) were injected over immobilized Sortilin at increasing concentrations. Sensorgrams obtained for Katana's peptides (see FIGS. 13 and 14) and Sortilin ligands (see FIGS. 15 and 16) clearly demonstrate direct interaction with immobilized Sortilin. Overall, the results indicate that both Katana peptide compounds interact with Sortilin with an affinity in the low nM range. The below Table shows the affinity constant KD that was calculated in nM using the Bia Evaluation software, from the sensorgrams obtained in FIGS. 13-16.

| | | Affinity |
|---|---|---|
| Family | Compound | (KD) |
| Katana Peptides Family 1 | KBP-106 | 46 |
| Katana Peptides Family 2 | KBP-201 | 3.3 |
| Sortilin ligands | RAP | 4.2 |
| Sortilin ligands | Neurotensin | 117 |

Based on these results Katana peptides of the second Katana peptide family (KB-P201) has a better affinity than one of the peptide of the first peptide family (KBP-106). Interestingly, both KBP-106 and KBP-201 have a better affinity for Sortilin than Neurotensin, a peptide well known to be a Sortilin ligand.

In summary, these new results show that peptides of the second family (KBP Family 2 peptide compounds) have a better affinity of Sortilin. Furthermore, it is also demonstrated that the peptide compounds of the present disclosure can interfere with TNF-α cell signalling events associated with inflammation.

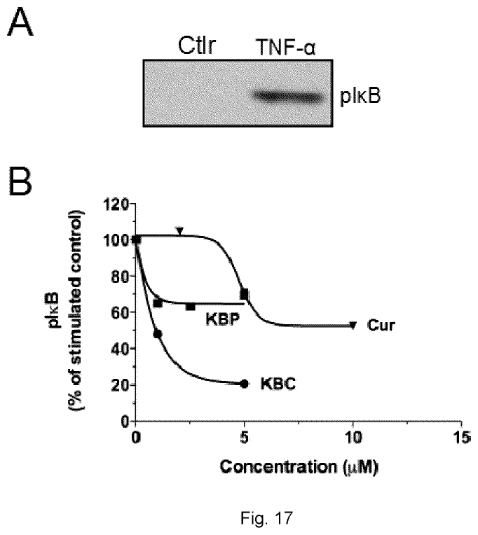

One of the inflammatory pathways induced by TNF-α leads to the phosphorylation IkB (see FIG. 1), a key protein in inflammation. It was observed that the addition of TNF-α to human HT-29 colon cancer cells triggered the phosphorylation of IkB (pIkB) (FIGS. 17A and 17B). Similar to COX-2 expression, the addition of KBC-201 caused a stronger inhibition of TNF-α-induced IkB phosphorylation than Curcumin alone. Interestingly, the addition of the peptide alone (KBP-201) reduced also the induced phosphorylation of IkB with a maximal effect of 40% at a lower concentration than Curcumin.

Regarding the the inhinition tests made in FIGS. 17A and 17B, (inhibition of TNF-α-induced IκB phosphorylation by Curcumin conjugate (KBC-201) in human HT-29 colon cancer cells), cells were pre-treated for 24 h with Curcumin (Cur), Curcumin peptide conjugate (KBC) or Katana peptide alone (KBP) in serum-free medium before the addition of 100 ng/mL TNF-α for 5 min. In FIG. 17A, immunodetection of IκB phosphorylation by TNF-α is shown and in FIG. 17B, the band intensities were analyzed by scanning densitometry using ImageJ software and the quantification is shown. For each sample, IκB phosphorylation level was corrected for GAPDH (a loading control) and normalized to those seen in TNF-α control (value=100%).

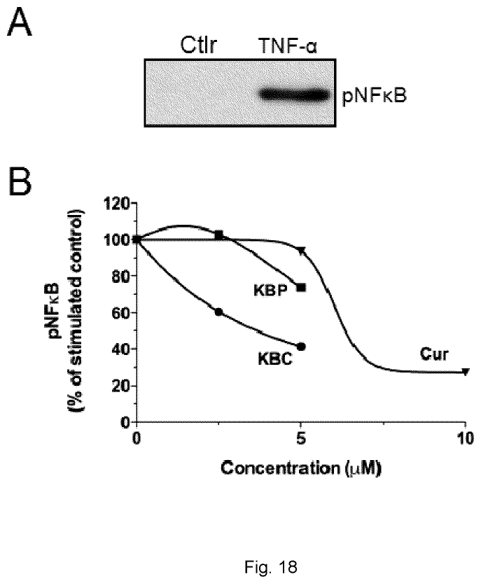

Curcumin conjugate (KBC-201) is more potent than free Curcumin to antagonize the phosphorylation of key pro-inflammatory proteins induced by TNF-α in the MDA-MB231 breast cancer cell model. Results presented in FIGS. 18A and 18B show now that the peptide alone (KBP-201) can reduce the TNF-α-induced NFκB phosphorylation by about 30%.

Regarding the the inhinition tests made in FIGS. 18A and 18B, (inhibition of TNF-α-induced NFκB phosphorylation by Curcumin conjugate (KBC-201) in human MDA-MB231 breast cancer cells), cells were pre-treated for 24 h with Curcumin (Cur), Curcumin conjugate KBC-201 (KBC) or Katana peptide KBP-201 alone (KBP) in serum-free medium before the addition of 100 ng/mL TNF-α for 5 min. In FIG. 18A, immunodetection of NFκB phosphorylation by TNF-α is shown. In FIG. 18B, the band intensities were analyzed by scanning densitometry using ImageJ software and the quantification is shown. For each sample, the phosphorylated NFκB/non phosphorylated NFκB ratio was normalized to those seen in TNF-α control (value=100%).

The embodiments of paragraphs [0046] to [00330] of the present disclosure are presented in such a manner in the present disclosure so as to demonstrate that every combination of embodiments, when applicable, can be made. These embodiments have thus been presented in the description in a manner equivalent to making dependent claims for all the embodiments that depend upon any of the preceding claims (covering the previously presented embodiments), thereby demonstrating that they can be combined together in all possible manners. For example, all the possible combinations, when applicable, between the embodiments of paragraphs [0046] to [00330] and the various aspects presented in paragraphs [007] to [0045] are hereby covered by the present disclosure.

### REFERENCES

1. Akil H., Perraud A., Melin C., Jauberteau M O., Mathonnet M. (2011) Fine-tuning roles of endogenous brain-derived neurotrophic factor, TrkB and sortilin in colorectal cancer cell survival. PloS One 6:e25097.
2. Al-Shawi R, Hafner A, Chun S, Raza S, Crutcher K, Thrasivoulou C, Simons P, Cowen T. (2007) ProNGF, sortilin, and age-related neurodegeneration. Ann N Y Acad Sci. 1119:208-15
3. Bradley J R. (2008) TNF-mediated inflammatory disease. J Pathol 2008; 214: 149-160
4. Carlo A S, Nykjaer A, Willnow T E. (2014) Sorting receptor sortilin—a culprit in cardiovascular and neurological diseases. J Mol Med (Berl). 92(9):905-11.
5. Dal Farra C., Sarret P., Navarro V., Botto J M., Mazella J., Vincent J P. (2001) Involvement of the neurotensin receptor subtype NTR3 in the growth effect of neurotensin on cancer cell lines. Int J Cancer 92:503-9
6. Fisher R, Pusztai L, Swanton C. (2013) Cancer heterogeneity: implications for targeted therapeutics. British Journal of Cancer. 108; 479-485.
7. Giorgi R R., Chile T., Bello A R., Reyes R., Fortes M A., Machado M C., Cescato V A., Musolino N R., Bronstein M D., Giannella-Neto D, et al. (2008) Expression of neurotensin and its receptors in pituitary adenomas. J Neuroendocrinol 20:1052-7
8. Hemmati S, Zarnani A H, Mahmoudi A R, Sadeghi M R, Soltanghoraee H, Akhondi M M, Tarahomi M, Jeddi-Tehrani M, Rabbani H. (2009) Ectopic Expression of Sortilin 1 (NTR-3) in Patients with Ovarian Carcinoma. Avicenna J Med Biotechnol. 1(2):125-31.
9. Lewin G R, Nykjaer A. (2014) Pro-neurotrophins, sortilin, and nociception. Eur J Neurosci. 39(3):363-74.
10. Mazella J, Vincent J P. (2006) Internalization and recycling properties of neurotensin receptors. Peptides. 27(10):2488-92.
11. Medzhitov R. (2008) Review Article: Origin and physiological roles of inflammation. Nature 454, 428-435.
12. Mortensen M. B., Kjolby M., Gunnersen S., Larsen J. V., Palmfeldt J., Falk E., Nykjaer A., and Bentzon J. F. (2014) JCI 124:5317-5322.
13. Sahebkar A, Cicero A F, Simental-Mendia L E, Aggarwal B B, Gupta S C. (2016) Curcumin downregulates human tumor necrosis factor-_levels: A systematic review and meta-analysis ofrandomized controlled trials. Pharmacol Res. 107:234-242.
14. Schmidt V, Willnow T E. (2016) Protein sorting gone wrong—VPS10P domain receptors in cardiovascular and metabolic diseases. Atherosclerosis. 245:194-9.
15. Truzzi F., Marconi A., Lotti R., Dallaglio K., French L E., Hempstead B L., Pincelli C. (2008) Neurotrophins and their receptors stimulate melanoma cell proliferation and migration. J Invest Dermatol 128:2031-40.
16. Vaegter C B., Jansen P., Fjorback A W., Glerup S., Skeldal S., Kjolby M., Richner M., Erdmann B., Nyengaard J R., Tessarollo L, et al. (2011) Sortilin associates with Trk receptors to enhance anterograde transport and neurotrophin signaling. Nat Neurosci 14:54-61
17. Vincent J P, Mazella J, Kitabgi P. (1999) Neurotensin and neurotensin receptors. Trends Pharmacol Sci. 20(7):302-309.
18. Wilson C M, Naves T, Saada S, Pinet S, Vincent F, Lalloue F, Jauberteau M O. (2014) The implications of sortilin/vps10p domain receptors in neurological and human diseases. CNS Neurol Disord Drug Targets. 13(8):1354-1365.
19. Wilson C M, Naves T, Al Akhrass H, Vincent F, Melloni B, Bonnaud F, Lalloue F, Jauberteau M O. (2016) A new role under sortilin's belt in cancer. Commun Integr Biol. 9(1):e1130192.
20. Yabe-Wada T., Matsuba S., Takeda K., Sato T., Suyama M., Ohkawa Y., Takai T., Shi H., Philpott C. C., Nakamura A. (2016) TLR signals posttranscriptionally regulate the cytokine trafficking mediator sortilin.

1. A peptide compound having at least 80% sequence identity to a compound chosen from compounds of formula (I), formula (II), formula (III), formula (IV), formula (V), formula (VI), formula (VII), formula (VIII), formula (IX), formula (X), formula (XI), formula (XII) and formula (XIII):
(I)
(SEQ ID NO: 1)
X₁X₂X₃X₄X₅GVX₆AKAGVX₇NX₈FKSESY
(II)
(SEQ ID NO: 2)
(X₉)ₙVX₁₀AKAGVX₁₁NX₁₂FKSESY
(III)
(SEQ ID NO: 3)
YKX₁₃LRRX₁₄APRWDX₁₅PLRDPALRX₁₆X₁₇L
(IV)
(SEQ ID NO: 4)
YKX₁₈LRR(X₁₉)ₙPLRDPALRX₂₀X₂₁L
(V)
(SEQ ID NO: 5)
IKLSGGVQAKAGVINMDKSESM
(VI)
(SEQ ID NO: 6)
IKLSGGVQAKAGVINMFKSESY
(VII)
(SEQ ID NO: 7)
IKLSGGVQAKAGVINMFKSESYK
(VIII)
(SEQ ID NO: 8)
GVQAKAGVINMFKSESY
(IX)
(SEQ ID NO: 9)
GVRAKAGVRNMFKSESY
(X)
(SEQ ID NO: 10)
GVRAKAGVRN(Nle)FKSESY
(XI)
(SEQ ID NO: 11)
YKSLRRKAPRWDAPLRDPALRQLL
(XII)
(SEQ ID NO: 12)
YKSLRRKAPRWDAYLRDPALRQLL
(XIII)
(SEQ ID NO: 13)
YKSLRRKAPRWDAYLRDPALRPLL
wherein
X₁, X₂, X₃, X₄, X₅, X₆, X₇, X₈, X₉, X₁₀, X₁₁, X₁₂, X₁₃, X₁₄, X₁₅, X₁₈ and X₁₉ are independently chosen from any amino acid;
X₁₆, X₁₇, X₂₀ and X₂₁ are independently chosen from Q, P, Y, I and L;
n is 0, 1, 2, 3, 4 or 5;
when X₉ is present more than once, each of said X₉ is independently chosen from any amino acid;
when X₁₉ is present more than once, each of said X₉ is independently chosen from any amino acid,
and wherein at least one protecting group and/or at least one labelling agent is optionally connected to said peptide compound at an N- and/or C-terminal end,
for use in treating inflammation.

2-5. (canceled)

6. The peptide compound of claim 1, wherein the peptide compound is represented by formula (V) and consists of the amino acid sequence of SEQ ID NO: 5.

7-10. (canceled)

11. The peptide compound of claim 1, wherein the peptide compound is represented by formula (X) and consists of the amino acid sequence of SEQ ID NO: 10.

12. The peptide compound of claim 1, wherein the peptide compound is represented by formula (XI) and consists of the amino acid sequence of SEQ ID NO: 11.

13-14. (canceled)

15. The peptide compound claim 1, wherein the peptide compound has at least 90% sequence identity to the compound chosen from compounds of formula (I), formula (II), formula (III), formula (IV), formula (V), formula (VI), formula (VII), formula (VIII), formula (IX), formula (X), formula (XI), formula (XII) and formula (XIII).

16. The peptide compound of claim 1, wherein the peptide compound comprises at least one protecting group that is acetyl or succinyl.

17. (canceled)

18. The peptide compound of claim 1, wherein the peptide compound is represented by Formula (XXXVIII), Formula (XXXIX), Formula (XXXX), Formula (XXXXI) or Formula (XXXXII):
(XXXVIII)
(SEQ ID NO: 14)
Acetyl-GVRAKAGVRNMFKSESY
(XXXIX)
(SEQ ID NO: 15)
Acetyl-GVRAKAGVRN(Nle)FKSESY
(XXXX)
(SEQ ID NO: 16)
Acetyl-YKSLRRKAPRWDAPLRDPALRQLL
(XXXXI)
(SEQ ID NO: 17)
Acetyl-YKSLRRKAPRWDAYLRDPALRQLL
(XXXXII)
(SEQ ID NO: 18)
Acetyl-YKSLRRKAPRWDAYLRDPALRPLL.

19-20. (canceled)

21. A conjugate compound having the formula of A-(B)ₙ,
wherein
n is 1, 2, 3 or 4;
A is a peptide compound as defined in claim 1, wherein said peptide compound is optionally protected by a protecting group; and
B is at least one therapeutic agent, wherein B is connected to A, optionally at a free amine of said peptide compound, at an N-terminal position of said peptide compound, at a free —SH of said peptide compound, or at a free carboxyl of said peptide compound,
for use in treating inflammation.

22. A conjugate compound having the formula of A-(B)ₙ,
wherein
n is 1, 2, 3 or 4;
A is a peptide compound as defined in claim 1, wherein said peptide compound is optionally protected by a protecting group; and
B is at least one therapeutic agent, wherein B is connected to A at a free amine of a lysine residue of said peptide compound, optionally via a linker, or at an N-terminal position of said peptide compound, optionally via a linker,
for use in treating inflammation.

23. (canceled)

24. The conjugate compound of claim 21, wherein the at least one therapeutic agent is an anti-inflammatory agent.

25. The conjugate compound of claim 24, wherein the anti-inflammatory agent is a phytochemical, a non-steroidal anti-inflammatory drug, a steroidal anti-inflammatory drug, an antileukotrine agent, a biologic agent or an immune-selective anti-inflammatory derivative (ImSAID).

26. The conjugate compound of claim 25, wherein the anti-inflammatory agent is a phytochemical chosen from curcumin, omega-3, white willow bark, green tea, catechins, pycnogenol, Boswellia serrata resin, resveratrol, Uncaria tomentosa, capsaicin, anthocyanins/anthocyanidins, flavanoids, olive oil compounds, chlorogenic acid and sulfopharaphane.

27. The conjugate compound of claim 25, wherein the anti-inflammatory agent is a non-steroidal anti-inflammatory drug chosen from Aspirin (Anacin, Ascriptin, Bayer, Bufferin, Ecotrin, Excedrin), Choline and magnesium salicylates (CMT, Tricosal, Trilisate), Choline salicylate (Arthropan), Celecoxib (Celebrex), Diclofenac potassium (Cataflam), Diclofenac sodium (Voltaren, Voltaren XR), Diclofenac sodium with misoprostol (Arthrotec), Diflunisal (Dolobid), Etodolac (Lodine, Lodine XL), Fenoprofen calcium (Nalfon), Flurbiprofen (Ansaid), Ibuprofen (Advil, Motrin, Motrin IB, Nuprin), Indomethacin (Indocin, Indocin SR), Ketoprofen (Actron, Orudis, Orudis KT, Oruvail), Magnesium salicylate (Arthritab, Bayer Select, Doan's Pills, Magan, Mobidin, Mobogesic), Meclofenamate sodium (Meclomen), Mefenamic acid (Ponstel), Meloxicam (Mobic), Nabumetone (Relafen), Naproxen (Naprosyn, Naprelan*), Naproxen sodium (Aleve, Anaprox), Oxaprozin (Daypro), Piroxicam (Feldene), Rofecoxib (Vioxx), Salsalate (Amigesic, Anaflex 750, Disalcid, Marthritic, Mono-Gesic, Salflex, Salsitab), Sodium salicylate (various generics), Sulindac (Clinoril), and Tolmetin sodium (Tolectin).

28. The conjugate compound of claim 25, wherein the anti-inflammatory agent is a steroidal anti-inflammatory drug chosen from Hydrocortisone type drugs, for example Hydrocortisone, methylprednisolone, prednisolone, prednisone, and triamcinolone (short- to medium-acting glucocorticoid), Acetonides for example Amcinonide, budesonide, desonide, fluocinolone acetonide, fluocinonide, halcinonide, and triamcinolone acetonide, Betamethasone type drugs, for example Beclometasone, betamethasone, dexamethasone, fluocortolone, halometasone, and mometasone, esters, for example: Halogenated esters (less labile) such as Alclometasone dipropionate, betamethasone dipropionate, betamethasone valerate, clobetasol propionate, clobetasone butyrate, fluprednidene acetate, and mometasone furoate, and Labile prodrug esters, such as Ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate.

29. The conjugate compound of claim 25, wherein the anti-inflammatory agent is a antileukotrine agent chosen from Leukotriene receptor antagonists, such as montelukast, zafirlukast, and pranlukast, and 5-lipoxygenase inhibitors, such as zileuton and Hypericum perforatum.

30. The conjugate compound of claim 25, wherein the anti-inflammatory agent is a biologic agent chosen from Rituximab, Abatacept, Tocilizumab, Etanercept, Adalimumab, Infliximab, Ankinra.

31. The conjugate compound of claim 25, wherein the anti-inflammatory agent is an ImSAID that is a SGP-T derivative.

32-52. (canceled)

53. A method of treating inflammation comprising administering to a subject in need thereof a therapeutically effective amount of at least one compound as defined in claim 21.

54. (canceled)

55. A method of treating inflammation in cells expressing Sortilin, comprising contacting said cells with at least one compound as defined in claim 21.

56. A method of inhibiting TNF-α-induced COX-2 expression in cells expressing Sortilin, comprising contacting said cells with at least one compound as defined in claim 21.

57-85. (canceled)


SEQUENCE LISTING (22 sequences)

SEQ ID NO: 1 - protein, 22 aa, Artificial Sequence
  Synthetic Construct
  MISC_FEATURE (1)..(5): Xaa can be any amino acid
  MISC_FEATURE (8)..(8): Xaa can be any amino acid
  MISC_FEATURE (14)..(14): Xaa can be any amino acid
  MISC_FEATURE (16)..(16): Xaa can be any amino acid
XXXXXGVXAKAGVXNXFKSESY

SEQ ID NO: 2 - protein, 23 aa, Artificial Sequence
  Synthetic Construct
  MISC_FEATURE (1)..(6): Xaa can be any amino acid and can be either present or absent
  MISC_FEATURE (9)..(9): Xaa can be any amino acid
  MISC_FEATURE (15)..(15): Xaa can be any amino acid
  MISC_FEATURE (17)..(17): Xaa can be any amino acid
XXXXXXGVXAKAGVXNXFKSESY

SEQ ID NO: 3 - protein, 24 aa, Artificial Sequence
  Synthetic Construct
  MISC_FEATURE (3)..(3): Xaa can be any amino acid
  MISC_FEATURE (7)..(7): Xaa can be any amino acid
  MISC_FEATURE (13)..(13): Xaa can be any amino acid
  MISC_FEATURE (22)..(23): Xaa can be either Gln, Pro, Tyr, Ile or Leu
YKXLRRXAPRWDXPLRDPALRXXL

SEQ ID NO: 4 - protein, 23 aa, Artificial Sequence
  Synthetic Construct
  MISC_FEATURE (3)..(3): Xaa can be any amino acid
  MISC_FEATURE (7)..(12): Xaa can be any amino acid and can be either present or absent
  MISC_FEATURE (21)..(22): Xaa can be either Gln, Pro, Tyr, Ile or Leu
YKXLRRXXXXXXPLRDPALRXXL

SEQ ID NO: 5 - protein, 22 aa, Artificial Sequence
  Synthetic Construct
IKLSGGVQAKAGVINMDKSESM

SEQ ID NO: 6 - protein, 22 aa, Artificial Sequence
  Synthetic Construct
IKLSGGVQAKAGVINMFKSESY

SEQ ID NO: 7 - protein, 23 aa, Artificial Sequence
  Synthetic Construct
IKLSGGVQAKAGVINMFKSESYK

SEQ ID NO: 8 - protein, 17 aa, Artificial Sequence
  Synthetic Construct
GVQAKAGVINMFKSESY

SEQ ID NO: 9 - protein, 17 aa, Artificial Sequence
  Synthetic Construct
GVRAKAGVRNMFKSESY

SEQ ID NO: 10 - protein, 17 aa, Artificial Sequence
  Synthetic Construct
  MISC_FEATURE (11)..(11): Xaa is Nle
GVRAKAGVRNXFKSESY

SEQ ID NO: 11 - protein, 24 aa, Artificial Sequence
  Synthetic Construct
YKSLRRKAPRWDAPLRDPALRQLL

SEQ ID NO: 12 - protein, 24 aa, Artificial Sequence
  Synthetic Construct
YKSLRRKAPRWDAYLRDPALRQLL

SEQ ID NO: 13 - protein, 24 aa, Artificial Sequence
  Synthetic Construct
YKSLRRKAPRWDAYLRDPALRPLL

SEQ ID NO: 14 - protein, 17 aa, Artificial Sequence
  Synthetic Construct
  MOD_RES (1)..(1): ACETYLATION
GVRAKAGVRNMFKSESY

SEQ ID NO: 15 - protein, 17 aa, Artificial Sequence
  Synthetic Construct
  MOD_RES (1)..(1): ACETYLATION
  MISC_FEATURE (11)..(11): Xaa is Nle
GVRAKAGVRNXFKSESY

SEQ ID NO: 16 - protein, 24 aa, Artificial Sequence
  Synthetic Construct
  MOD_RES (1)..(1): ACETYLATION
YKSLRRKAPRWDAPLRDPALRQLL

SEQ ID NO: 17 - protein, 24 aa, Artificial Sequence
  Synthetic Construct
  MOD_RES (1)..(1): ACETYLATION
YKSLRRKAPRWDAYLRDPALRQLL

SEQ ID NO: 18 - protein, 24 aa, Artificial Sequence
  Synthetic Construct
  MOD_RES (1)..(1): ACETYLATION
YKSLRRKAPRWDAYLRDPALRPLL

SEQ ID NO: 19 - protein, 23 aa, Homo sapiens
KCLRREAPRWDAPLRDPALRQLL

SEQ ID NO: 20 - protein, 23 aa, Rattus norvegicus
KCLRKKTPRWDILLRDPAPRPLL

SEQ ID NO: 21 - protein, 23 aa, Mus musculus
KCLRKKIPRWDMFLRDPVPRPLL

SEQ ID NO: 22 - protein, 13 aa, Homo sapiens
  MISC_FEATURE (1)..(1): Xaa can be any amino acid
XLYENKPRRPYIL